(19) United States
(12) Patent Application Publication
(10) Pub. No.: US 2025/0163513 A1
(43) Pub. Date: May 22, 2025

(54) METHODS OF TREATMENT FOR HPV MALIGNANCIES

(71) Applicant: The University of North Carolina at Chapel Hill, Chapel Hill, NC (US)

(72) Inventor: Travis Schrank, Chapel Hill, NC (US)

(21) Appl. No.: 18/839,605

(22) PCT Filed: Feb. 22, 2023
(86) PCT No.: PCT/US2023/063005

§ 371 Date: Aug. 19, 2024

Related U.S. Application Data
(60) Provisional application No. 63/313,056, filed on Feb. 23, 2022.

(51) Int. Cl. C12Q 1/6886; C12Q 1/70

(52) U.S. Cl. CPC C12Q 1/6886; C12Q 1/708; C12Q 2600/106; C12Q 2600/156; C12Q 2600/158

(57) ABSTRACT

This invention relates to human papilloma virus (HPV) positive cancers such as HPV⁺ squamous cell carcinoma of the oropharynx (OPSCC). This invention further relates methods of determining treatment regimens, methods of stratifying prognosis from treatment of HPV positive cancers, methods of determining suitability for de-escalation of treatment of HPV positive cancers, and methods of treating HPV positive cancers.

### STATEMENT OF PRIORITY

This application claims the benefit, under 35 U.S.C. § 119 (e), of U.S. Provisional Application No. 63/313,056, filed on Feb. 23, 2022, the entire contents of which are incorporated by reference herein.

### STATEMENT OF GOVERNMENT SUPPORT

This invention was made with government support under Grant Number DE029241 awarded by the National Institutes of Health. The government has certain rights in the invention.

### STATEMENT REGARDING ELECTRONIC FILING OF A SEQUENCE LISTING

A Sequence Listing in XML format, entitled 5470-905WO_ST26.xml, 1,103,014 bytes in size, generated on Feb. 22, 2023 and filed herewith, is hereby incorporated by reference in its entirety for its disclosures.

### FIELD OF THE INVENTION

This invention relates to human papilloma virus (HPV) positive cancers such as HPV⁺ squamous cell carcinoma of the oropharynx (OPSCC). This invention further relates methods of determining treatment regimens, methods of stratifying prognosis from treatment of HPV positive cancers, methods of determining suitability for de-escalation of treatment of HPV positive cancers, and methods of treating HPV positive cancers.

### BACKGROUND OF THE INVENTION

HPV-positive (HPV+) squamous cell carcinoma of the oropharynx (OPSCC) is the most prevalent HPV-associated malignancy in the United States and is primarily caused by HPV16. HPV+ OPSCC has surpassed cervical cancer in incidence and is the most commonly diagnosed malignancy caused by HPV in the USA (Pan et al. Cancers Head Neck. 2018; 3). HPV+ OPSCC has an improved prognosis compared to non-HPV OPSCC, however, treatment can carry significant, lifelong therapeutic toxicity. While the concept of de-escalation of therapy represents an effort to limit morbidity while preserving tumor control, there are few available tools to select appropriate patients with favorable prognosis.

The present invention overcomes previous shortcomings in the art by providing methods of stratifying risk prognosis, determining treatment regimens, and determining suitability for de-escalated therapy for HPV-associated malignancies such as OPSCC.

### SUMMARY OF THE INVENTION

One aspect of the present invention provides a method of determining a treatment regimen for a subject having human papillomavirus (HPV) positive (HPV+) cancer (e.g., OPSCC) or a subject at risk for or suspected to have or develop HPV+ cancer (e.g., OPSCC), comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) determining the prognosis of the subject upon treatment to the cancer, wherein a low divergence of the sequence information of the detected HPV virus genome and/or genome product as compared to the reference HPV viral genome identifies the subject as a candidate for standard (e.g., “therapeutic”) treatment for OPSCC, and wherein a greater divergence identifies the subject as a suitable candidate for de-escalated (e.g., “sub-therapeutic”) treatment for the cancer.

Another aspect of the present invention provides a method of stratifying prognosis from treatment of human papillomavirus (HPV) positive cancer in a subject having human papillomavirus (HPV) positive (HPV+) cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) stratifying the prognosis of the subject upon treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer.

Another aspect of the present invention provides a method of determining suitability for de-escalation of treatment of human papillomavirus (HPV) positive cancer in a subject having human papillomavirus (HPV) positive (HPV+) cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) determining the prognosis of the subject upon treatment to the cancer, wherein a greater divergence identifies the subject as a suitable candidate for de-escalated (e.g., “sub-therapeutic”) treatment for the cancer.

Another aspect of the present invention provides a method of de-escalating treatment of human papillomavirus (HPV) positive cancer in a subject having human papillomavirus (HPV) positive (HPV+) cancer or a subject at risk for or suspected to have or develop HPV+ cancer and undergoing standard (e.g., “therapeutic,” “escalated”) treatment of the cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer; and f) treating the subject identified as having reduced risk of poor prognosis with de-escalated treatment as compared to standard treatment for the cancer.

Another aspect of the present invention provides a method of treating human papillomavirus (HPV) positive (HPV+) cancer in a subject having human papillomavirus (HPV) positive (HPV+) cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer; and f) treating the subject identified as having reduced risk of poor prognosis with de-escalated treatment as compared to standard (e.g., “therapeutic”) treatment for the cancer.

In some embodiments, the sequence information of the HPV viral genome and/or genome product in the sample comprises RNA and/or DNA sequence information.

### BRIEF DESCRIPTION OF THE DRAWINGS

FIG. 1 shows a schematic of HPV16 positivity assignment and cohort composition. FIG. 1 panel A. Histogram of sequencing reads mapping to HPV16, normalized to human reads. Dashed line-Threshold for HPV16 positivity applied. FIG. 1 panel B. Normalized reads mapping to HPV16 versus a library of 336 other HPV genomes included in the ViFi package. C. Flow diagram illustrating cohort construction for genotypic and clinical analyses.

FIG. 2 shows bar graphs quantifying sequence conservation by viral gene and characteristics of sub-clonal polymorphisms. FIG. 2 panel A. Conservation of Viral Genes. All clonal polymorphisms relative to the HPV16A1 reference were examined. The ratio of missense to synonymous SNPs were compared between the gene in question and all other viral genes. Significance based on Wilcoxon Rank-sum test. FIG. 2 panel B. Conservation of Viral Genes based on Uncommon Polymorphisms. Clonal polymorphisms were examined if present in less than 25% of cases examined. The ratio of missense to synonymous uncommon SNPs were compared between the gene in question and all other viral genes. Significance based on Wilcoxon Rank-sum test. FIG. 2 panel C. Distribution of non-synonymous polymorphisms amongst HPV16 viral genes. Significance based on chi-squared test. FIG. 2 panel D. Proportion of SNPs by predicted effect. Significance based on chi-squared test. FIG. 2 panel E. Proportion of potentially APOBEC related SNPs in the HPV16 viral genome, compared between clonal and sub-clonal SNPs. Significance based on chi-squared test. *** P-value<1*10\hat{ }-3.

FIG. 3 shows data plots analyzing HPV16 Genotypes by Sub-lineage. FIG. 3 panel A. Heatmap of common non-synonymous polymorphisms in the HPV16 genome. Columns-patient tumor samples. Sub-lineage—the nearest HPV16 sub-lineage reference sequence by the JC69 sequence distance. Number of Non-synon. Poly.—Number of non-synonymous polymorphisms relative to the HPV16 A1 reference. Relative Viral Copy Number-Viral copy-number relative to human genomic material. Defined as Log 2 (HPV16 Reads/Human Reads). Common Polymorphism—The 15 most common non-synonymous polymorphisms relative to HPV16 A1 are listed in order of decreasing prevalence. FIG. 3 panel B. Kaplan-Meier Plot of Recurrence-free Survival, comparing sub-lineage A1 to others. P-value represents log-rank test. HR-estimated hazard ratio with 95% confidence interval. FIG. 3 panel C. Proportion of Cases Stratified by AJCC8 Clinical Staging. Significance based on chi-squared test. FIG. 3 panel D. Tobacco Smoke Exposure. Significance based on Wilcoxen Rank-sum test. FIG. 3 panel E. Predicted Viral Proteomic Neo-Antigenicity. Neo-Antigenicity was estimated by the number of viral peptides with <350 nM affinity for MHC, based on the patients HLA subtype. Significance based on Wilcoxen Rank-sum test. FIG. 3 panel F. Human Genomic Copy-number Variant Burden. Number of copy number events as identified by the SynthEx pipeline. Significance based on Wilcoxon Rank-sum test. * P-value <5*10\hat{ }-2. NS-not significant. A1—HPV16 genome assigned to HPV16-A1 sub-lineage based on nearest JC69 distance. Other-HPV16 genome assigned to sub-lineage other than HPV16-A1 based on nearest JC69 distance. FIG. 3 panel G. HPV16 Sub-lineages for sequenced HPV16+ tumors from the oropharynx and uterine cervix. FIG. 3 panel H. Common HPV16 Non-synonymous polymorphisms in sequenced HPV16+ tumors from the oropharynx and uterine cervix. FIG. 3 panel I. Proportion of Tumors with HPV16 Deep Copy Loss in E2 or a large loss (involving >10% of the viral genome). Chi-squared test. P-value<5*10\hat{ }-2.

FIG. 4 shows a schematic of viral phylogeny and related bar graphs quantifying tumor Genomic and Patient Factors Stratified by Maximum Parsimony Phylogeny of OPSCC HPV16 Viral Genomes. FIG. 4 panel A. Maximum Parsimony Phylogeny of HPV16 genomes. Color—Above or below the median number of non-synonymous clonal polymorphisms in the HPV16 genome relative to the A1 reference sequence. Tip point size/color—Total number of non-synonymous clonal polymorphisms in the HPV16 genome relative to the A1 reference sequence. FIG. 4 panel B. Kaplan-Meier Plot of Recurrence-free Survival, comparing viral clades indicated in Panel A. P-value represents log-rank test. HR-estimated hazard ratio with 95% confidence interval. FIG. 4 panel C. Proportion of Cases Stratified by AJCC8 Clinical Staging in near and far clades. Significance based on chi-squared test. FIG. 4 panel D. Tobacco Smoke Exposure for near and far clades. Significance based on Wilcoxon Rank-sum test. FIG. 4 panel E. Predicted Viral Proteomic Neo-Antigenicity for near and far clades. Neo-Antigenicity was estimated by the number viral peptides with <350 nM affinity for MHC, based on the patients HLA subtype. Significance based on Wilcoxon Rank-sum test. FIG. 4 panel F. Human Genomic Copy-number Variant Burden. Number of copy number events as identified by the SynthEx pipeline. Significance based on Wilcoxon Rank-sum test. * P-value<5*10\hat{ }-2.

FIG. 5 shows data plots regarding the origins of Environmental and Intra-tumoral Genomic Diversity of Oncogenic HPV16 in HNSCC. FIG. 5 panels A and B. Mutational Signature Analysis of Uncommon HPV16 Non-synonymous Clonal (Environmental) Polymorphisms. All non-synonymous polymorphisms identified in less than 25% of tumors were included. Non-negative matrix decomposition of all included SNPs was performed based on the COSMIC signatures V2, as implemented by the DeconstructSigs R package. FIG. 5 panel A. Tumors in the viral clade near the HPV16-A1 reference sequence. FIG. 5 panel B. Viral genomes distal to relative to HPV16-A1. FIG. 5 panels C-E. Estimating HPV16 Sub-clonality with Binomial Mixture Clustering Analysis. FIG. 5 panel C. Scatter plot of Sub-clone Defining Viral SNPs. VAF—variant allele frequency. FIG. 5 panel D. Bar Plot of frequency of tumors by number of HPV16 sub-clonal populations. Populations defined by VAF Groups defined by binomial mixture clustering with BIC analysis as implemented in the R Canopy package.

FIG. 6 shows a schematic of viral phylogeny and data plots HPV16 Viral Genotyping and Integration Analysis by RNA Sequencing. FIG. 6 panel A. Maximum Parsimony Phylogeny of HPV16 genomes from the DNA sequencing cohort. Links-Connect known (DNAseq) genotype, with nearest neighbor genotype assigned from RNAseq, for 13 patients with available DNA and RNA sequencing data. Color—Viral clade as assigned in FIG. 4. Tip point size—Number of RNAseq cases assigned at a neighbor to the indicated DNAseq case. FIG. 6 panels B-G. Kaplan-Meier Plot of Recurrence-free Survival, comparing viral clades vs. viral integration status, stratified by tobacco smoke exposure. P-value represents log-rank test. HR—estimated hazard ratio with 95% confidence interval. FIG. 6 panel H. Annotated heat map of HPV16 viral gene expression. Columns—tumor samples, organized by E6E7/E5E2 ratio. Viral Clade—as assigned in panel A. Integrated—Assigned integration status based in E6E7/E5E2 ratio. Split Reads—Presence of detectable split read—pairs mapping to both the HPV16 and human genome. FIG. 6 panels I-K. Genomic and clinical features of the <=10 pack-year smoking exposure sub-group. FIG. 6 panel I. HPV16 Integration status-based in E6E7/E5E2 ratio Significance based on Chi-squared test. FIG. 6 panel J. AJCC8 summary stage. Significance based on Chi-squared test. FIG. 6 panel K. Tobacco Smoke Exposure. Significance based on Wilcoxon Rank-sum test. * P-value<5*10\hat{ }-2. NS—not significant. Near-HPV16 viral clade nearest to the HPV16-A1 sub-lineage based on JC69 distance. Far—HPV16 viral clade distal to the HPV16-A1 sub-lineage based on JC69 distance.

FIG. 7 shows Kaplan-Meier Plots of Recurrence-free Survival, comparing viral clades as assigned in FIG. 4, stratified by tobacco smoke exposure. FIG. 7 panel A. Patients from initial DNA sequencing cohort with no more than 30 pack-years of tobacco smoke exposure. FIG. 7 panel B. Patients initial DNA sequencing cohort with no more than 10 pack-years of tobacco smoke exposure.

FIG. 8 shows a Waterfall Plot of somatic variants identified. Genes area displayed in order of decreasing frequency of somatic alteration. Near HPV16-A1 Clade. Variants from 18 patients from initial DNA sequencing cohort assigned to the near A1 clade. Divergent from HPV16-A1 Clade. Variants from 19 patients from initial DNA sequencing cohort assigned to the clade divergent from A1, as in FIG. 4. Color-predicted variant effect of SNPs and small INDELs. Structural variants were not analyzed. No statistical differences could be detected between the two groups.

FIG. 9 shows average sequencing coverage depth by genomic locus of the HPV16 genome.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.” With respect to the terms “comprising”, “consisting essentially of”, and “consisting of”, where one of these three terms is used herein, the presently disclosed subject matter can include the use of either of the other two terms.

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or fewer (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or fewer additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “sequence identity,” as used herein, has its standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to their default values. The parameters are dynamic values and are established by the program itself depending upon the composition of the particular sequence of interest and the composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-BLAST-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences that contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations, relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or modified nucleotide bases. The term “nucleotide sequence” or “nucleic acid sequence” refers to both the sense and antisense strands of a nucleic acid, either as individual single strands or in the duplex. The term “ribonucleic acid” (RNA) is inclusive of RNAi (inhibitory RNA), dsRNA (double stranded RNA), siRNA (small interfering RNA), shRNA (short/small hairpin RNA), mRNA (messenger RNA), miRNA (micro-RNA), tRNA (transfer RNA, whether charged or discharged with a corresponding acylated amino acid), long non-coding RNA (lncRNA), ribosomal RNA (rRNA), small nuclear RNA (snRNA), small nucleolar RNA (snoRNA) and CRNA (complementary RNA), and the term “deoxyribonucleic acid” (DNA) is inclusive of cDNA and genomic DNA and DNA-RNA hybrids.

As used herein, “HPV” refers to any human papillomavirus, including HPV strains 16, 18, 31, 33, 35, 39, 45, 51, 52, 56, 58, 59, 66, and 68. In particular embodiments, the HPV strain is HPV-16. As used herein, “positive for HPV” or “HPV-positive” means the subject and/or sample has been infected with the HPV virus.

As used herein, “p16” refers to the p16 gene, as well as gene products encoded and/or derived therefrom.

As used herein, OPSCC refers to “oropharyngeal squamous cell carcinoma,” a cancer commonly referred to as “throat cancer” and/or “tonsil cancer,” derived from the squamous epithelium lining the middle part of the pharynx (the oropharynx), extending vertically from the soft palate to the superior area of the hyoid bone and including the base and posterior of the tongue, the tonsils, soft palate, and posterior and lateral pharyngeal walls. OPSCC can be categorized into HPV-positive and HPV negative cancer, sometimes also referred to as p16 and/or HPV/p16-positive and negative cancer. p16 (also known as p16^{INK4a} and/or CDKN2A) is a cyclin-dependent kinase inhibitor and tumor suppressor protein encoded by the CDKN2A gene commonly used as a biomarker for epithelial neoplasia and optionally used as a proxy for HPV infection.

As used herein, a “genome product” refers to any material produced from the expression of a genome such as a viral genome, including but not limited to DNA, RNA (e.g., mRNA, miRNA, etc.), RNP, and proteins.

“Amino acid sequence” and terms such as “peptide”, “polypeptide”, and “protein” are used interchangeably herein, and are not meant to limit the amino acid sequence to the complete, native amino acid sequence (i.e., a sequence containing only those amino acids found in the protein as it occurs in nature) associated with the recited protein molecule.

As used herein, the terms “sequence information,” “nucleic acid sequencing information,” “nucleic acid sequencing data,” “sequence information,” “nucleic acid sequence,” “genomic sequence,” “genome sequence,” “genetic sequence,” “fragment sequence,” “nucleic acid sequencing read,” “read,” and the like denote any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc. Further examples of sequencing technologies include those described in, e.g., U.S. PGPub US/2020/0051663 and US/2020/0002747, incorporated herein by reference.

As used herein, the term “dataset” refers to a collection of related sets of information, i.e., data, attained from experimental or computational analyses, comprising any type of data, including but not limited to nucleic acid sequences or amino acid sequences (i.e., “sequence information”). The dataset may be screened and/or otherwise searched for particular data of interest depending on variable parameters as defined by each particular dataset. In some embodiments, the dataset is a nucleic acid dataset, i.e., a dataset comprising nucleic acid sequences. The source material (e.g., healthy subject(s) and/or patient(s)) may be alternatively referred to as a database, a repository, a reference group, a cohort, a library, or any similar terminology understood in the art.

In some embodiments, a dataset set of invention may be a collection of reference viral genomes, also referred to as a reference library. In some embodiments, a reference library may comprise two or more viral genomes (e.g., two or more reference viral genomes). The viral genomes may be known viral genomes and/or newly isolated and/or sequenced viral genomes, and as such a reference library may comprise the sequence information of known viral genomes and/or of newly sequenced viral genomes including any viral genomes not yet known and/or sequences. In some embodiments, a reference library of the present invention is a pre-existing reference library, de novo generated from newly sequenced viral genomes, or any combination thereof.

As used herein, the term “biomarker” can mean any chemical or biological entity that is produced by cells (e.g., cells of the subject), or substances that are produced by cells that might be then chemically modified by extracellular enzymes, free radicals produced by cells of the body and/or other naturally occurring processes and that is found, for example, in the saliva, urine, blood, vaginal secretion, tears, feces, sputum, hair, nails, skin, wound fluid, nasal swab, lymph, perspiration, oral mucosa, vaginal mucosa, or the anus, or in serum or plasma obtained from blood. In some embodiments, a biomarker of the present invention may be a polymorphism.

The terms “polymorphic” and “polymorphism” as used herein (e.g. genetic variation), refer to variation in the sequence of a gene in the genome or the encoded amino acid sequence thereof amongst a population, such as allelic variations and other variations that arise or are observed. Thus, a polymorphism refers to the occurrence of two or more genetically determined alternative sequences or alleles in a population. These differences can occur in coding and non-coding portions of the genome, and can be manifested or detected as differences in nucleic acid sequences and/or gene expression, including, for example, transcription, processing, translation, transport, protein processing, trafficking, DNA synthesis, expressed proteins, protein modifications, RNA expression modification, DNA and RNA methylation, regulatory factors that alter gene expression and DNA replication, other gene products or products of biochemical pathways or in post-translational modifications, and any other differences manifested amongst members of a population in genomic nucleic acid or organelle nucleic acids.

A polymorphic site or polymorphic position refers to a site in the nucleic acid sequence at which divergence occurs. Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also include different mendelian alleles for a gene. A single nucleotide polymorphism (SNP) refers to a polymorphism that arises as the result of a single base change (i.e., a single nucleotide position), such as an insertion, deletion or change in a base. The term “genotype” refers to a description of the alleles of a gene or genes contained in an individual or a sample.

When compared to a reference genome, a particular genotype (e.g., of a genome in an individual or sample) may be referred to as having more or less divergence from the reference sequence, relative to the number of polymorphisms comprise in the genome as compared to the reference genome. Divergence as compared to a reference genome may be expressed in qualitative and/or quantitative terms, such as but not limited to, as a number of polymorphisms (e.g., SNPs, non-synonymous (i.e., coding) polymorphisms, synonymous polymorphisms, all polymorphisms, or any combination thereof), relative and/or absolute copy number of any one or more polymorphisms as compared to the reference genome, as well as a direct and/or normalized comparison of nucleic acid sequence information of any one or more genome product, genome fragment, and/or total genome as compared to the reference genome (e.g., percent sequence identity, sequence identity score, various sequence distance scores, and the like). As used herein, relative terms of divergence such as a “low” divergence or a “high” divergence may correspond with a quantifiable amount of divergence. For example, in some embodiments a low divergence may comprise a divergence of less than or equal to 100, 99.9, 99.5, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25, 0.10, 0.05, 0.01% from a reference genome, in terms of any of the above quantified sequence information variables (e.g., number of polymorphisms, copy number of polymorphisms, percent sequence identity, sequence identity score, various sequence distance scores and/or other sequence information). In some embodiments a high divergence may comprise a divergence of greater than or equal to 100, 99.9, 99.5, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25, 0.10, 0.05, 0.01% from a reference genome, in terms of any of the above quantified sequence information variable. In some embodiments, a low divergence may comprise a divergence of less than or equal to a particular predetermined threshold for a relevant variable (e.g., number of polymorphisms, copy number of polymorphisms, percent sequence identity, sequence identity score, various sequence distance scores and/or other sequence information). In some embodiments, a “high” divergence may comprise a divergence of greater than or equal to such a predetermined threshold.

As used herein, the term “sample” is used in its broadest sense. In one sense, it is meant to include a specimen from a biological source. Biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject. Thus, in the methods of this invention, the sample can be any biological fluid or tissue that can be used in a method of this invention, including but not limited to, serum, plasma, blood, saliva, semen, lymph, cerebrospinal fluid, prostatic fluid, urine, sputum, oral mucosa, nasal mucosa, duodenal fluid, gastric fluid, skin, endothelium, biopsy material from a salivary gland, biopsy material of a parotid gland, biopsy material of other glands of the mouth, secretions of the salivary gland, secretions of the parotid gland, secretions of other glands of the mouth, joint fluid, body cavity fluid, tear fluid, anal secretions; vaginal secretions, perspiration, whole cells, cell extracts, tissue, biopsy material, aspirates, exudates, slide preparations, fixed cells, tissue sections, etc.

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human. The terms “subject” and “patient” are in some embodiments used interchangeably herein, such as but not limited to in reference to a human subject or patient.

A “subject in need” of the methods of the invention can be any subject known or suspected to have an HPV+ cancer such as OPSCC to which the methods of the present invention disclosed herein may provide beneficial health effects, or a subject having an increased risk of developing the same.

As used herein the term “control” refers to a comparative sample and/or other reference source for a control subject.

The terms “administering” and “administration” of a treatment to a subject include any route of introducing or delivering to a subject a compound to perform its intended function. Administration can be carried out by any suitable route, including orally, intranasally, parenterally (intravenously, intramuscularly, intraperitoneally, intracisternally, intrathecally, intraventricularly, or subcutaneously), or topically. Administration includes self-administration and administration by another.

By the terms “treat,” “treating,” and “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

As used herein, the terms “prevent,” “prevents,” and “prevention” (and grammatical equivalents thereof) refer to a delay in the onset of a disease or disorder or the lessening of symptoms upon onset of the disease or disorder. The terms are not meant to imply complete abolition of disease and encompass any type of prophylactic treatment that reduces the incidence of the condition or delays the onset and/or progression of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

In some embodiments, a subject of the present invention may be administered standard (e.g., “therapeutic”) treatment for a relevant disorder of the invention, such as but not limited to, OPSCC. Standard therapeutic treatment regimens for HPV+ cancers such as OPSCC are known in the art and can be readily implemented by the skilled artisan (e.g., an oncologist). As used herein, the term “escalate” and the like (e.g., “escalated,” “escalation, etc.”) may be used to refer to standard (e.g., “therapeutic” level) treatment for the disorder, and/or to refer to a return to standard/therapeutic level treatment following prior lack of treatment (naïve) or other (e.g., de-escalated, alternative, supratherapeutic, etc.) treatment.

In some embodiments, a subject of the present invention may be administered “de-escalated” (e.g., “sub-therapeutic”) treatment for a relevant disorder of the invention, such as but not limited to, OPSCC. As used herein, the term “de-escalate” and the like (e.g., “de-escalated,” de-escalating,” etc.) refers to a reduction (e.g., to a decrease or diminishment in the specified dose or activity of at least about 15%, 25%, 35%, 40%, 50%, 60%, 75%, 80%, 90%, 95% or more) or elimination of a standard (e.g., “therapeutic”) treatment of the disorder (e.g., to a “sub-therapeutic” level of treatment). De-escalation may be inclusive of any one or more treatment(s) targeted to the disorder, in any combination, as well as de-escalation of an entire treatment regimen. In some embodiments, the de-escalation may be a reduction or elimination of, for example, radiation therapy, chemotherapy, immunotherapy, surgery, and/or intubation of the subject.

### Methods

This invention relates to human papilloma virus (HPV) positive cancers such as HPV⁺ squamous cell carcinoma of the oropharynx (OPSCC). This invention further relates methods of determining treatment regimens, methods of stratifying prognosis from treatment of HPV positive cancers, methods of determining suitability for de-escalation of treatment of HPV positive cancers, and methods of treating HPV positive cancers.

HPV subtype 16 (HPV16) is the most common high-risk HPV causing OPSCC. There are four main variant lineages (A, B, C, D) and at least 10 known sub-lineages (A1, A2, A3, A4, B1, B2, C, D1, D2, D3) of HPV16. Formerly, these sub-lineages were geographically termed European (A1-3), Asian (A4), African-1 (B), African-2 (C), and North-American/Asian-American (D1-3). Lineages are defined by 1-10% differences and sub-lineages defined by 0.5-1% differences in the L1 (capsid protein) sequence (Smith B, Chen Z, Reimers et al. PLOS ONE. 2011; 6(6): e21375; Burk et al. Virology. 2013; 445(1-2):232-243; Mirabello et al. J Natl Cancer Inst. 2016; 108(9)).

Along with OPSCC and other anogenital cancers, HPV16 is also the most common high-risk HPV associated with cervical cancer. HPV16 sub-lineage classification of cervical cancer has indicated that though lineage A1 is most prevalent, non-A HPV16 lineages (B/C/D) are associated with a higher risk of precancer and cancer (Burk 2013; Mirabello 2016). HPV16 lineage D variants are thought to be associated with the highest rate of persistent infection and progression to cervical cancer compared with other variants.

HPV16 viral oncoproteins E6 and E7 bind to p53 and pRb, respectively, and contribute to carcinogenesis and inhibition of apoptosis and entry of cell into the S phase. The E6 L83V polymorphism has been associated with infection persistence and progression of cervical carcinoma within the A1-3 sub-lineages. However, few studies have examined HPV16+ OPSCC.

RNA sequencing data have been used to determine HPV positivity and viral genome integration (Cancer Genome Atlas Network. Comprehensive genomic characterization of head and neck squamous cell carcinomas. Nature. 2015; 517(7536):576-582. HPV integration in OPSCC has been reported to correlate with genomic methylation and tumor mutational burden (Parfenov et al. Proc Natl Acad Sci USA. 2014; 111(43):15544-15549), genomic instability (Akagi et al. Genome Res. 2014; 24(2):185-199), HPV gene expression (Walline et al. Mol Cancer Res MCR. 2016; 14(10):941-952), and tumor immune landscape and survival (Koneva et al. Mol Cancer Res MCR. 2018; 16(1):90-102).

HPV+ OPSCC has an improved prognosis compared to non-HPV OPSCC, however, treatment can carry significant, lifelong therapeutic toxicity. De-escalation of therapy represents an effort to limit morbidity while preserving tumor control (Cheraghlou et al. Cancer. 2018; 124(4):717-726; Chera et al. Cancer. 2018; 124(11):2347-2354; Chera et al. Clin Cancer Res Off J Am Assoc Cancer Res. 2019; 25(15):4682-4690; Marur et al. J Clin Oncol Off J Am Soc Clin Oncol. 2017; 35(5):490-497), however, initial results have been mixed and there are few available tools to select appropriate patients with favorable prognosis.

Thus, one aspect of the invention relates to a method of determining a treatment regimen for a subject having HPV positive (HPV+) cancer (e.g., OPSCC) or a subject at risk for or suspected to have or develop HPV+ cancer (e.g., OPSCC), comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) determining the prognosis of the subject upon treatment to the cancer, wherein a low divergence of the sequence information of the detected HPV virus genome and/or genome product as compared to the reference HPV viral genome identifies the subject as a candidate for standard (e.g., “therapeutic”) treatment for OPSCC, and wherein a greater divergence identifies the subject as a suitable candidate for de-escalated (e.g., “sub-therapeutic”) treatment for the cancer.

Another aspect of the invention relates to a method of stratifying prognosis from treatment of HPV+ cancer in a subject having HPV+ cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) stratifying the prognosis of the subject upon treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer.

Another aspect of the invention relates to a method of determining suitability for de-escalation of treatment of human papillomavirus (HPV) positive cancer in a subject having human papillomavirus (HPV) positive (HPV+) cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; and e) determining the prognosis of the subject upon treatment to the cancer, wherein a greater divergence identifies the subject as a suitable candidate for de-escalated (e.g., “sub-therapeutic”) treatment for the cancer.

In some embodiments, a low divergence identifies the subject as not a suitable candidate for de-escalated treatment for the cancer; e.g., identifies the subject as having elevated risk of poor prognosis from de-escalated treatment to cancer.

In some embodiments, methods of the present invention may further comprise treating the subject identified as a suitable candidate for de-escalated treatment and/or having reduced risk of poor prognosis with de-escalated treatment to the cancer.

Another aspect of the invention relates to a method of de-escalating treatment of human papillomavirus (HPV) positive cancer in a subject having human papillomavirus (HPV) positive (HPV+) cancer or a subject at risk for or suspected to have or develop HPV+ cancer and undergoing standard (e.g., “therapeutic,” “escalated”) treatment of the cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer; and f) treating the subject having reduced risk of poor prognosis with de-escalated treatment as compared to standard treatment for the cancer.

Another aspect of the invention relates to a method of treating human papillomavirus (HPV) positive (HPV+) cancer in a subject having human papillomavirus (HPV) positive (HPV+) cancer or a subject at risk for or suspected to have or develop HPV+ cancer, comprising: a) obtaining a sample from the subject; b) detecting a level of expression of HPV virus genome and/or genome product in the sample; c) obtaining sequence information of the HPV viral genome and/or genome product in the sample; d) comparing the sequence information obtained in c) to a reference HPV viral genome; e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated (e.g., “sub-therapeutic”) treatment to the cancer, and wherein a greater divergence categorizes the subject as having reduced risk of poor prognosis from de-escalated treatment to the cancer; and f) treating the subject identified as having reduced risk of poor prognosis from de-escalated treatment with de-escalated treatment as compared to standard (e.g., “therapeutic”) treatment for the cancer.

The sequence information of the HPV viral genome and/or genome product of the invention may be obtained by any suitable method known in the art, including but not limited to standard nucleic acid sequencing methods as will be apparent to one skilled in the art upon review of the present disclosure. Many methods are known in the art for obtaining sequence information and are within the scope of the presently disclosed subject matter, including but not limited to whole genome (e.g., viral genome) sequencing, Sanger sequencing, next-generation sequencing (NGS), high-throughput sequencing, pyrosequencing (“454” sequencing), sequencing by ligation (SOLID sequencing), nanopore sequencing, polony sequencing, massively parallel signature sequencing (MPSS), iIllumina sequencing, metagenomic sequencing, polymerase chain reaction (PCR) amplification sequencing, target enrichment sequencing, RNA sequencing (RNAseq), chromatin-immunoprecipitation sequencing (chIP-seq), and shotgun sequencing. In some embodiments, obtaining sequence information of the HPV viral genome and/or genome product in the sample may comprise obtaining RNA and/or DNA sequence information.

In some embodiments of the methods of the invention, the comparing step (e.g., of comparing the sequence information obtained to a reference HPV viral genome) may comprise identifying one or more polymorphism(s) of the detected HPV virus genome and/or genome product and comparing the identified one or more polymorphism(s) to polymorphisms of the reference HPV viral genome, wherein a divergence of 17 or fewer (e.g., 17, 16, 15, 14, 13, 12, 11, 10, 9, 8, 7, 6, 5, 4, 3, 2, 1, or fewer, or any value or range therein) polymorphisms (e.g., single-nucleotide polymorphisms (SNPs) and/or non-synonymous polymorphisms) of the detected HPV virus genome and/or genome product as compared to the reference HPV viral genome categorizes the subject as having elevated risk of poor prognosis from de-escalated treatment to the cancer, and wherein a divergence of 18 or more (e.g., 18, 19, 20, 21, 22, 23, 24, 25, 26, 27, 28, 29, 30 or more, or any value or range therein) polymorphisms (e.g., single-nucleotide polymorphisms (SNPs) and/or non-synonymous polymorphisms) to the reference HPV viral genome categorizes the subject as having reduced risk of poor prognosis from de-escalated treatment to the cancer.

In some embodiments, a low diverge of the detected HPV virus genome and/or genome product may be a divergence of 17 (e.g., 17, 16, 15, 14, 13, 12, 11, 10, 9, 8, 7, 6, 5, 4, 3, 2, 1, or fewer, or any value or range therein) polymorphisms of any kind as compared to the reference HPV viral genome, e.g., SNPs, non-synonymous (i.e., coding) polymorphisms, synonymous polymorphisms, or any combination thereof. In some embodiments, a low diverge of the detected HPV virus genome and/or genome product may be a divergence of 8 (e.g., 8, 7, 6, 5, 4, 3, 2, 1, or fewer, or any value or range therein) non-synonymous polymorphisms of the detected HPV virus genome and/or genome product as compared to the reference HPV viral genome.

In some embodiments of the methods of the invention, the comparing step (e.g., of comparing the sequence information obtained to a reference HPV viral genome) may comprise performing statistical and/or probabilistic analyses which may indicate a relative relationship to (“near” or “far”) the reference HPV viral genome. Upon review of the present disclosure, those skilled in the art will be familiar with numerous statistical and/or probabilistic analyses and variations thereof that can be useful for carrying out the methods of the presently disclosed subject matter. For example, in some embodiments, a nearest neighbor analysis may be useful for carrying out the methods of the invention. As used herein, the term “nearest neighbor,” nearest neighbor distribution,” “nearest neighbor function,” and the like refer to a mathematical function that is defined in relation to a point process, representable as randomly positioned points in time, space or both. Nearest neighbor functions are defined with respect to some point in the point process as being the probability distribution of the distance from this point to its nearest neighboring point in the same point process, and accordingly describe the probability of another point existing within some distance of a particular point.

Accordingly, in some embodiments of the methods of the invention, the comparing step (e.g., of comparing the sequence information obtained to a reference HPV viral genome) may comprise determining a nearest neighbor distribution of the detected HPV virus genome and/or genome product as compared to the reference HPV viral genome, wherein a nearest neighbor distribution of the sequence information of the detected HPV virus genome and/or genome product less than a predetermined threshold as compared to (“grouping with”) the reference HPV viral genome categorizes the subject as having elevated risk of poor prognosis from de-escalated treatment to the cancer, and wherein a nearest neighbor distribution equal to or greater than the pre-determined threshold as compared to (e.g., “grouping outside” of) the reference HPV viral genome categorizes the subject as having reduced risk of poor prognosis from de-escalated treatment to the cancer.

In some embodiments of the methods of the invention, the comparing step (e.g., of comparing the sequence information obtained to a reference HPV viral genome) may comprise calculating a sequence distance (e.g., identity score and/or % sequence identity) of the sequence information of the detected HPV virus genome and/or genome product as compared to the reference HPV viral genome, wherein a sequence distance equal to or less than a pre-determined threshold of the detected HPV virus genome and/or genome product as compared to (e.g., “near” to) the reference HPV viral genome categorizes the subject as having elevated risk of poor prognosis from de-escalated treatment to the cancer, and wherein a sequence distance greater than the pre-determined threshold of the detected HPV virus genome and/or genome product as compared to (e.g., “far” from) the reference HPV viral genome categorizes the subject as having reduced risk of poor prognosis from de-escalated treatment to the cancer.

In some embodiments of the methods of the invention, the comparing step (e.g., of comparing the sequence information obtained to a reference HPV viral genome) may comprise any combination of the above methods of comparing.

In some embodiments, the pre-determined threshold for the nearest neighbor distribution and/or the sequence distance is determined by a phylogenetic analysis (e.g., maximum parsimony phylogenetic analysis) of the reference HPV viral genome, wherein the HPV viral genome is a reference library (e.g., a de novo reference library, e.g., a pre-existing reference library) comprising a multitude of (e.g., at least two or more) reference HPV viral genomes.

In some embodiments, the phylogenetic analysis establishes at least two or more groups, wherein at least one group includes a HPV A1 reference genome (e.g., the “in-group,” the “high risk group”), and wherein at least one group does not include a HPV A1 reference genome (e.g., the “out-group,” the “low risk group”).

In some embodiments, the low risk group may include but is not limited to any one of the following HPV genomes (e.g., HPV reference genomes) which sequences are provided in the SEQUENCES section, in any combination thereof: “UNCseq1628” SEQ ID NO:13, “UNCseq1770” SEQ ID NO:23, “UNCseq2162” SEQ ID NO:58, “UNCseq1750” SEQ ID NO: 22, “UNCseq1864” SEQ ID NO:30, “UNCseq1867” SEQ ID NO:31, “UNCseq2234” SEQ ID NO:62, “UNCseq2491” SEQ ID NO:80, “UNCseq2539” SEQ ID NO:82, “UNCseq2012” SEQ ID NO:48, “UNCseq2554” SEQ ID NO:83, “UNCseq2771” SEQ ID NO: 93, “UNCseq1796” SEQ ID NO:25, “UNCseq0733” SEQ ID NO: 1, “UNCseq2051” SEQ ID NO: 52, “UNCseq2789” SEQ ID NO: 102, “UNCseq2106” SEQ ID NO:56, “UNCseq1742” SEQ ID NO:20, “UNCseq1140” SEQ ID NO:4, “UNCseq1849” SEQ ID NO: 28, “UNCseq1980” SEQ ID NO:41, “UNCseq2005” SEQ ID NO:45, “UNCseq2105” SEQ ID NO:55, “UNCseq1360” SEQ ID NO:6, “UNCseq1525” SEQ ID NO:7, “UNCseq1891” SEQ ID NO:32, “UNCseq1662” SEQ ID NO: 14, “UNCseq1924” SEQ ID NO: 36, “UNCseq2292” SEQ ID NO:66, “UNCseq1994” SEQ ID NO:43, “UNCseq2007” SEQ ID NO:46, “UNCseq1834” SEQ ID NO:26, “UNCseq2783” SEQ ID NO:100, “UNCseq2786” SEQ ID NO:101, “UNCseq1693” SEQ ID NO:66, “UNCseq2033” SEQ ID NO: 51, “UNCseq0848” SEQ ID NO:2, “UNCseq2249” SEQ ID NO:63, “UNCseq2794” SEQ ID NO: 106, “UNCseq2393” SEQ ID NO: 72, “UNCseq2576” SEQ ID NO:84, “UNCseq2795” SEQ ID NO: 107, “UNCseq1697” SEQ ID NO: 17, “UNCseq1938” SEQ ID NO: 38, “UNCseq1991” SEQ ID NO:42, GenBank® Accession Nos. AF536179.1 (HPV16 A2), HQ644236.1 (HPV16 A3), AF534061.1 (HPV16 A4), AF536180.1 (HPV16 B1), KU053915.1 (HPV16 B2), HQ644298.1 (HPV16 B3), KU053914.1 (HPV16 B4), AF472509.1 (HPV16 C1), HQ644244.1 (HPV16 C2), KU053920.1 (HPV16 C3), KU053925.1 (HPV16 C4), HQ644257.1 (HPV16 D1), AY686579.1 (HPV16 D2), AF402678.1 (HPV16 D3), KU053931.1 (HPV16 D4), and/or any of SEQ ID NOs: 108-123, in any combination thereof.

In some embodiments, the high risk group may include any HPV genomes (e.g., HPV reference genomes) which sequences are provided in the SEQUENCES section other than those listed above, in any combination thereof, including but not limited to HPV16 A1.

In some embodiments, the high risk group is defined as inclusive of any viral genome with a divergence of 17 or fewer (e.g., 17, 16, 15, 14, 13, 12, 11, 10, 9, 8, 7, 6, 5, 4, 3, 2, 1, or fewer, or any value or range therein) polymorphisms (e.g., single-nucleotide polymorphisms (SNPs) and/or non-synonymous polymorphisms) as compared to the reference HPV viral genome.

In some embodiments, the high risk group is defined as inclusive of any viral genome with a divergence of 8 or fewer (e.g., 17, 16, 15, 14, 13, 12, 11, 10, 9, 8, 7, 6, 5, 4, 3, 2, 1, or fewer, or any value or range therein) non-synonymous polymorphisms as compared to the reference HPV viral genome.

The sequence distance score may be calculated by any suitable method known in the art. In some embodiments, the sequence distance score is calculated according to JC69 or F81 sequence distances.

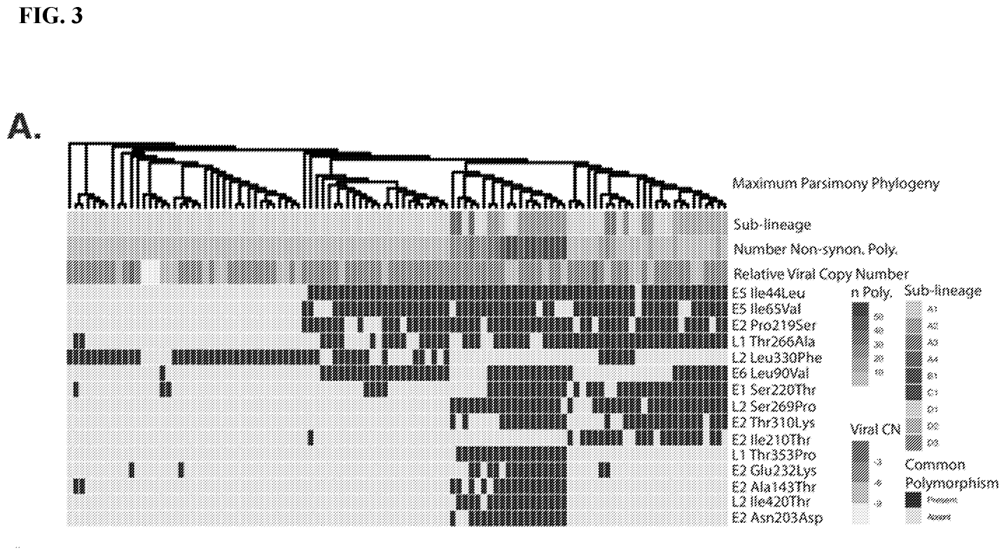

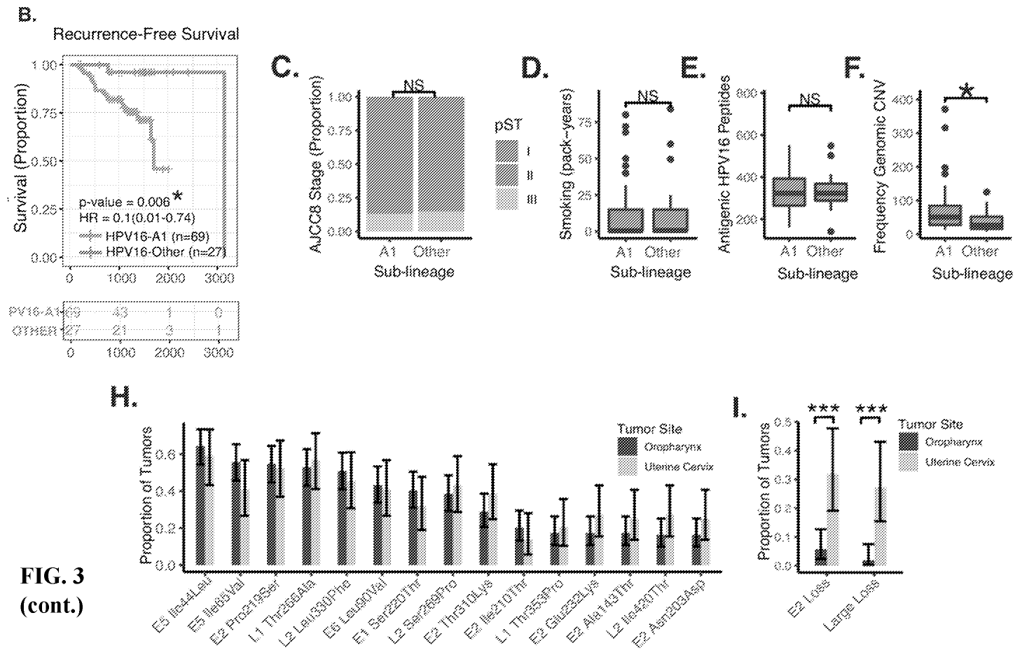

Polymorphisms relevant to the present invention include any known or as yet discovered polymorphisms between a relevant HPV genome and the reference HPV viral genome of HPV16 A1 strain GenBank® Accession No. NC_001526.4. In some embodiments, the one or more polymorphism(s) of the invention comprise all polymorphisms, single nucleotide polymorphisms (SNPs), non-synonymous (i.e., coding) polymorphisms, synonymous polymorphisms, or any combination thereof. For example, in some embodiments, the one or more polymorphism(s) comprise non-synonymous polymorphisms and/or SNPs. In some embodiments, the one or more polymorphism(s) comprises non-synonymous polymorphisms. In some embodiments, the one or more polymorphism(s) comprise any one or more of E5 I44L, E5 I65V, E2 P219S, L1 T266A, L2 L330F, E6 L90V, E1 S220T, L2 S269P, E2 T310K, E2 I210T, L1 T353P, E2 E232K, E2 A143T, L2 I420T, or E2 N203D polymorphisms, any one of the polymorphisms of FIG. 3, any other polymorphism disclosed herein, and any combination thereof.

In some embodiments of the methods of the present invention, the identifying step (e.g., identifying one or more polymorphism(s) of the detected HPV virus genome and/or genome product and comparing the identified polymorphisms to polymorphisms of the reference HPV viral genome) comprises identifying the presence of one or more of a subset of specific polymorphisms in the detected HPV virus genome and/or genome product and comparing the identified polymorphisms to the presence of the subset of specific polymorphisms in the reference HPV viral genome, wherein a divergence of less than about 8 (e.g., about 5, 6, 7, 8, 9, 10 or more) polymorphisms of the detected HPV virus genome and/or genome product as compared to the reference HPV viral genome categorizes the subject as having elevated risk of poor prognosis from de-escalated treatment to the cancer, and wherein a divergence of greater than about 8 polymorphisms (e.g., about 5, 6, 7, 8, 9, 10 or more) to the reference HPV viral genome categorizes the subject as having reduced risk of poor prognosis from de-escalated treatment to the cancer. The subset of specific polymorphisms may comprise any known polymorphism of interest and/or a polymorphism not yet known or discovered. In some embodiments, the subject of specific polymorphisms may include, but is not limited to, E5 I44L, E5 I65V, E2 P219S, L1 T266A, L2 L330F, E6 L90V, E1 S220T, L2 S269P, E2 T310K, E2 I210T, L1 T353P, E2 E232K, E2 A143T, L2 I420T, or E2 N203D polymorphisms, any one of the polymorphisms of FIG. 3, any other polymorphism disclosed herein, and any combination thereof.

The polymorphisms described above and elsewhere herein are denoted by [protein name, residue position relative to that protein], wherein the numbering corresponds to the relevant amino acid sequence encoded by the reference HPV viral genome of HPV16 A1 strain GenBank® Accession No. NC_001526.4. However it would be readily understood by one of ordinary skill in the art that the equivalent amino acid positions in other HPV virus amino acid sequences or other HPV virus genome sequences can be readily identified and employed in the utilization of this invention.

Standard therapeutic treatment regimens for HPV+ cancers such as OPSCC are known in the art and can be readily implemented by the skilled artisan (e.g., an oncologist). De-escalation (“sub-therapeutic”) of standard (“therapeutic”) may comprise a reduction of any standard treatment for a particular disorder which reduces an undesirable side effect of the standard therapeutic dose of said treatment. In some embodiments, de-escalated treatment comprises treating the subject with no and/or a reduced amount (e.g., 5%, 10%, 15%, 20%, 30%, 40%, 50%, 60%, 70%, 80%, 90% reduced amount or any value or range therein) as compared to standard treatment.

In some embodiments, de-escalated treatment of HPV+ OPSCC comprises treating the subject with no and/or a reduced amount (e.g., 5%, 10%, 15%, 20%, 30%, 40%, 50%, 60%, 70%, 80%, 90% reduced amount or any value or range therein) as compared to standard treatment of one or more treatments including but not limited to radiation therapy, chemotherapy, immunotherapy, surgery, and/or intubation of the subject.

In some embodiments, de-escalated treatment to the cancer comprises reducing the effective amount of chemotherapy administered to the subject (e.g., reducing the effective amount to about 5% to about 30% of the effective amount of chemotherapy; e.g., wherein de-escalated treatment to OPSCC comprises treating the subject with a sub-therapeutic amount (e.g., about 5% to about 30% of the effective amount) of chemotherapy.

In some embodiments, de-escalated treatment to the cancer comprises reducing the effective amount of radiation therapy administered to the subject (e.g., reducing the effective amount to about 5% to about 30% of the effective amount or radiation therapy; e.g., wherein de-escalated treatment to OPSCC comprises treating the subject with a sub-therapeutic amount (e.g., about 5% to about 30% of the effective amount) of radiation therapy.

In some embodiments, de-escalated treatment to the cancer (e.g., HPV+ cancer, e.g., HPV+ OPSCC) comprises not performing surgery for removal of cancerous and/or precancerous tissue on the subject.

In some embodiments, the subject is receiving and/or has previously received standard (e.g., “therapeutic,” “escalated”) treatment for the cancer (e.g., HPV+ cancer, e.g., HPV+ OPSCC).

In some embodiments, the subject is not receiving and/or has not previously received standard (e.g., “therapeutic,” “escalated”) treatment for the cancer (i.e., wherein the subject is treatment naïve).

In some embodiments, the subject is newly diagnosed has having cancer (e.g., HPV+ cancer, e.g., HPV+ OPSCC).

The HPV virus genome and/or genome product in the sample may comprise the genome and/or any genome product of any human papillomavirus, including but not limited to HPV strains 16, 18, 31, 33, 35, 39, 45, 51, 52, 56, 58, 59, 66, and 68. In some embodiments, the HPV virus genome and/or genome product in the sample comprises the genome and/or a genome product of a strain of HPV16 (e.g., sub-lineage(s) HPV16 A1, HPV16 A2-3, HPV16B1-4, HPV16 C1-4, and/or HPV16 D1-4).

The reference HPV viral genome may be any HPV viral genome of use as a reference for the utilization of the methods of this invention. In some embodiments, the reference HPV viral genome comprises a “high risk” HPV viral genome (e.g., an HPV viral genome encompassed in the high risk group, per phylogenetic analysis).

In some embodiments, the reference HPV viral genome comprises one or more reference HPV viral genomes (e.g., a reference library including but not limited to a de novo prepared reference library and/or a pre-existing reference library, e.g., UNCseq database).

In some embodiments, the reference HPV viral genome comprises the viral genome(s) of HPV strain GenBank® Accession No. NC_001526.4 (HPV16 A1, also numerated as K02718.1), AF536179.1 (HPV16 A2), HQ644236.1 (HPV16 A3), AF534061.1 (HPV16 A4), AF536180.1 (HPV16 B1), KU053915.1 (HPV16 B2), HQ644298.1 (HPV16 B3), KU053914.1 (HPV16 B4), AF472509.1 (HPV16 C1), HQ644244.1 (HPV16 C2), KU053920.1 (HPV16 C3), KU053925.1 (HPV16 C4), HQ644257.1 (HPV16 D1), AY686579.1 (HPV16 D2), AF402678.1 (HPV16 D3), KU053931.1 (HPV16 D4), any one of the sequences provided in the SEQUENCES section or any combination thereof.

In some embodiments, the reference HPV viral genome comprises, consists essentially of, or consists of the viral genome of HPV16 A1 strain GenBank® Accession No. NC_001526.4.

In some embodiments, the sample comprises a biopsy sample, blood sample, saliva sample, oral washing sample, and/or a direct tumor brushing/swabbing sample.

In some embodiments, the cancer is any HPV+ carcinoma, such as but not limited to HPV+ throat cancer and/or cervical cancer. In some embodiments, the cancer is HPV+ oropharyngeal squamous cell carcinoma (OPSCC).

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: Comprehensive Viral Genotyping Reveals Prognostic Viral Phylogenetic Groups in HPV16 Associated Squamous Cell Carcinoma of the Oropharynx

Briefly, in order to develop prognostic biomarkers to identify appropriately low-risk patients for reduced treatment intensity in patients with HPV-positive (HPV+) squamous cell carcinoma of the oropharynx (OPSCC), targeted DNA sequencing including all HPV16 open reading frames was performed on tumors from 104 patients with HPV16+ OPSCC treated at a single center. Genotypes closely related to HPV16-A1 were associated with increased numbers of somatic copy-number variants in the human genome. Genotypes divergent from HPV16-A1 were strongly associated with favorable recurrence-free survival as compared to HPV16-A1 (or similar genotype); this finding was independent of tobacco smoke exposure. Total RNA sequencing was performed on a second cohort of 89 HPV16+ OPSCC cases. HPV16 genotypes divergent from HPV16-A1 were again validated in this independent cohort, to be strongly prognostic of improved RFS in patients with moderate (less than 30 pack-years) or low (no more than 10 pack-years) of tobacco smoke exposure. Genotypes divergent from HPV16-A1 were also associated with lower rates of viral integration in tumors from patients with low tobacco smoke exposure. Sequence divergence from the HPV16-A1 reference sequence to be strongly correlated with improved recurrence-free survival in patients with moderate or low tobacco smoke exposure in two independent cohorts.

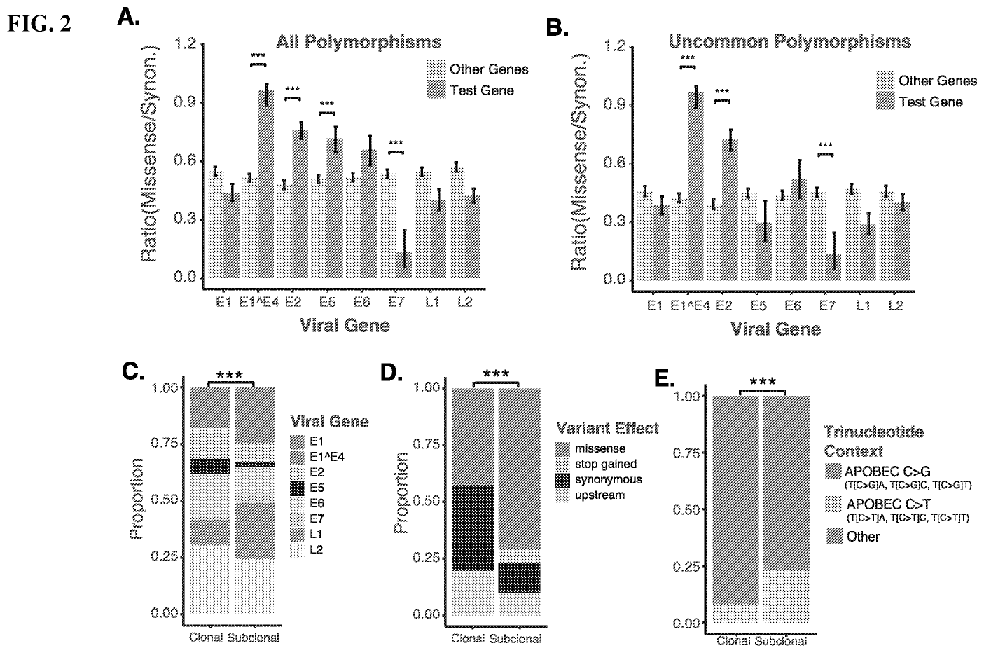

In detail, to examine the diversity of HPV16 oncogenic genotypes promoting OPSCC in the study cohort, polymorphisms with variant allele frequency >0.9 were considered to be clonal. Based on clonal polymorphisms alone, HPV16 coding genotypes were highly diverse, with 93 distinct protein coding HPV16 genomes amongst the 104 tumors examined. To assess selective pressure for protein sequence conservation, the ratio of coding to synonymous clonal sequence variants were considered. All polymorphisms relative to the HPV16 A1 reference were considered, as well as uncommon polymorphisms, defined as polymorphisms identified in less than 25% of tumors. Based on these metrics, E1\hat{ }E4 and E2 demonstrated the least conservation amongst all viral genes (FIG. 2 panels A-B). Consistent with expectations from the uterine cervical carcinoma literature, E7 demonstrated a high degree of conservation with few nonsynonymous polymorphisms (Mirabello et al. Cell. 2017; 170(6):1164-1174.e6; Arroyo-Mühr et al. Br J Cancer. 2018; 119(9):1163-1168).

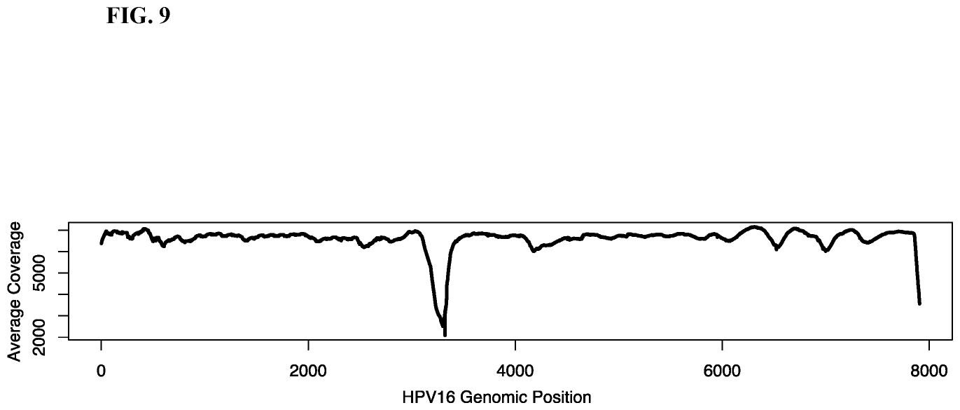

The viral sequencing data had an average depth of coverage of the HPV16 genome of ˜8,000, enabling analysis of sub-clonal genomic variants (FIG. 9). Despite the robust detection of sub-clonal variants, the bulk of genomic diversity was clonal with 1295 clonal coding (missense) polymorphisms vs. 144 sub-clonal coding polymorphisms. From 104 tumors, there were 285 unique (only present in one tumor) clonal non-synonymous variants. Sub-clonal variants demonstrated modest but detectable differences in distribution amongst HPV genes and were much more likely to cause protein-coding changes (FIG. 2 panels C-D). Considering prior reports supporting the relative importance of APOBEC-mediated mutagenesis in HPV+ OPSCC, APOBEC was investigated as a potential driver of HPV16 polymorphisms in OPSCC. Interestingly, polymorphisms corresponding to APOBEC deamination targets represented a minority of all clonal HPV polymorphisms identified (9.8%). However, 35.4% of sub-clonal variants (VAF <90%) were potentially APOBEC related (FIG. 2 panel E). This is consistent with expectations from analysis of cancerous and pre-malignant lesion of the uterine cervix, where APOBEC has been found to induce a minority of the lineage-defining SNPs, but sub-clonal APOBEC related variants are highly associated with viral clearance and failure of lesions to progress to invasive carcinoma (Revathidevi et al. Cancer Lett. 2021; 496:104-116; Zhu et al. Nat Commun. 2020; 11(1):886).

### HPV16 Sub-Lineage, Common Polymorphisms, and Viral Copy Loss:

HPV16 sub-lineage was also defined by the nearest HPV16 sub-lineage reference sequence in sequence space as determined by the Jukes and Cantor (JC69) (Jukes and Cantor. CHAPTER 24-Evolution of Protein Molecules. In: Munro H N, ed. Mammalian Protein Metabolism. Academic Press; 1969:21-132). Sixteen sub-lineages were queried based on contemporary studies (FIG. 3 panel A). HPV16-A1 genotype accounted the majority of (71%) tumors investigated. Eleven other tumors (13%) harbored A2-3 HPV16 and sub-lineages D1-3 were also represented by 11 tumors (13%). Maximum parsimony phylogenetic analysis based on all clonal SNPs grouped HPV16 oncogenic genomes, resulted in groups that were highly related in terms of HPV16 sub-lineage, common non-synonymous polymorphisms, and sequence divergence from HPV16 were all highly correlated.

Review of patient factors identified two patients presented with distant metastases and were treated with palliative intent; therefore 96 patients were available for analysis of recurrence-free-survival (RFS). The A1 sub-lineage was highly associated with poor recurrence-free survival (FIG. 3 panel B). This association persisted in a multivariate regression model including non-HPV16A1 genotype (HR=0.09, 95% CI=0.01-0.73, p-value=0.02), early vs. advanced AJCC8 summary stage (HR=1.4, 95% CI=0.38-4.8, p-value=0.64), and smoking exposure greater than 10 pack-years (HR=1.8, 95% CI=0.66-4.8, p-value=0.25).

Despite known associations of clinical stage and tobacco smoke exposure with outcome, there was no detectable association to viral sub-lineage (FIG. 3 panels D and E). Hypothesizing that some viral proteomes are more immunogenic, the number of high affinity (<325 nM) viral peptide/MHC interactions that would be expected given the individual patient's viral genotype and MHC subtype were estimated. No difference in antigenicity was identified between peptides encoded by the A1 vs. other sub-lineages (FIG. 3 panel E). Analysis of other thresholds and rank-based metrics of MHC peptide affinity also failed to yield and identifiable relationship to HPV16 genotype. The total number of copy-altered regions identified in the human genome (ref 32). The A1 sub-lineage correlated with the number of genomic CNVs (p=0.006, Wilcoxen Rank-sum test). Follow-up time, race, patient age, patient sex, treatment modality, and anatomic site of tumor origin were also examined, stratified according to HPV16 sub-lineage, (Table 1). There was a trend towards more white patients in the A1, high-risk group. Follow up time was longer in the non-A1, low-risk group.

The prevalence of common coding polymorphisms in HPV+ OPSCC was compared to a population matched (same center and time period) cohort of 44 HPV16+ uterine cervical squamosa cell carcinomas (UCSCC), sequenced with the same technique. The prevalence of common (clonal) coding polymorphisms were similar between HPV+ OPSCC and uterine cervical carcinoma (FIG. 3 panel H). There was a trend toward Non-A1 HPV16 sub-lineages being more common in the uterine cervical carcinoma (FIG. 3 panel G).

In 14/104 tumors, deep copy loss (depth of coverage <1% of that of tumor matched average E7 coverage) of a portion of the HPV genome was noted (excluding the non-coding hypervariable region 3150-3351). These losses, however, represented only 1.2% of all potential genomic space (tumor HPV bases) interrogated. In only 2 of 104 cases (1.9%), genomic regions with deep loss accounted for more than 10% of the viral genome (75%, 47%). These large genomic losses are likely to be related to (clonal) genomic integration of the HPV16 viral genome. Although integration events can be present as sub-clonal genomic variation, deep loss of large segments of the HPV16 genome was quite uncommon in HPV+OPSCC (FIG. 2 panel I). Similarly, only 6 of 104 (5.7%) tumors harbored deep losses of E2 of any size. Fourteen of 44 (32%) HPV16 positive tumors of the uterine cervix sequenced with the same strategy had deep loss involving E2. The difference in proportion of tumors harboring E2 loss between HPV16+ OPSCC (5.7%) and HPV16+UCSCC (32%) was significant (p<1*10\hat{ }-4, Chi squared test) (FIG. 2 panel I). Large scale losses (>10% of the viral genome) were also more common in UCSCC (p<1*10\hat{ }-4, Chi squared test) (FIG. 2 panel I).

### Phylogenetic Analysis:

Based on the clonal SNPs, relative to the HPV16-A1 reference, full HPV16 genotypes were reconstructed for each tumor. This approach may bias the few (n=2) cases with large areas of deep copy loss as being closer to the A1 reference sequence, as A1 reference is assumed for uncovered bases. To organize the sequence diversity, a maximum parsimony phylogenetic model was implemented. Common non-synonymous polymorphisms were highly correlated with the substructure of the phylogenetic tree. The most common protein-coding polymorphisms are displayed in FIG. 3 panel A. Relative viral copy number was estimated based on the ratio of HPV16 to human reads observed. Although relative viral copy number had a wide range (>1000 fold), this was not associated with phylogeny, HPV16 sub-lineage, or recurrence-free survival by any threshold examined.

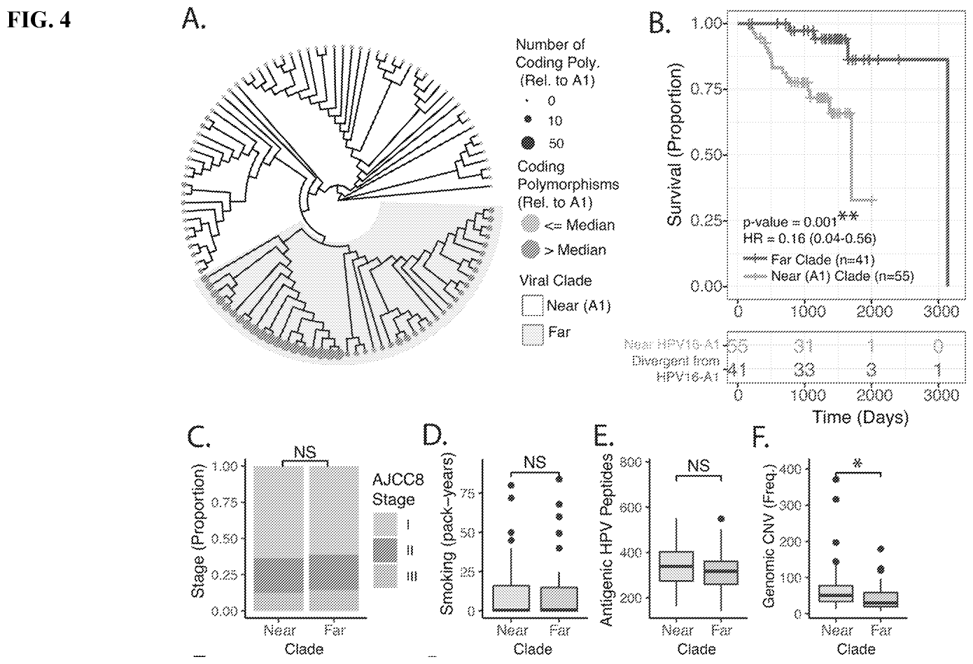

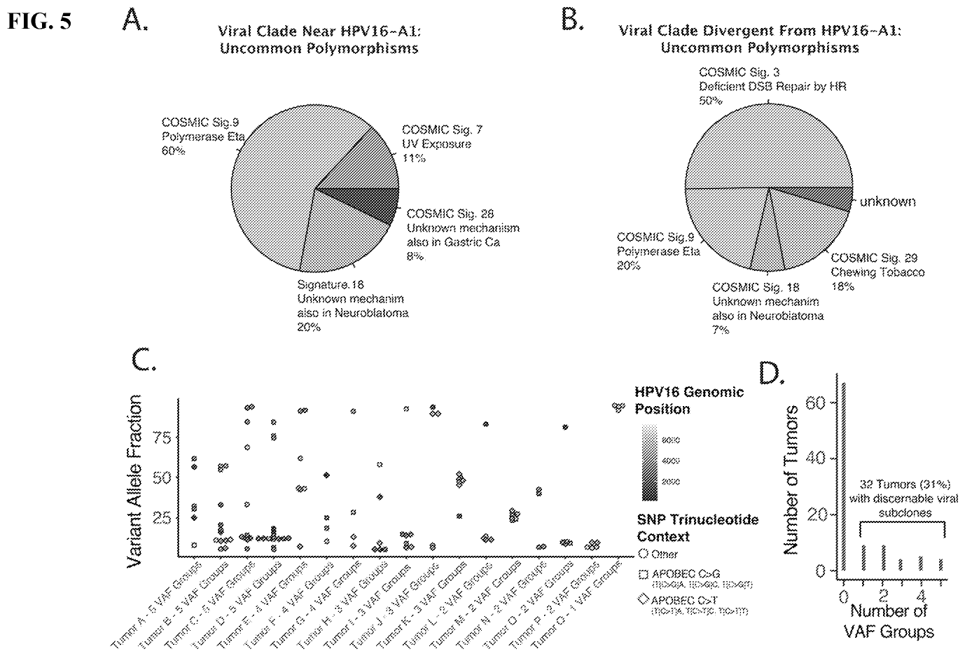

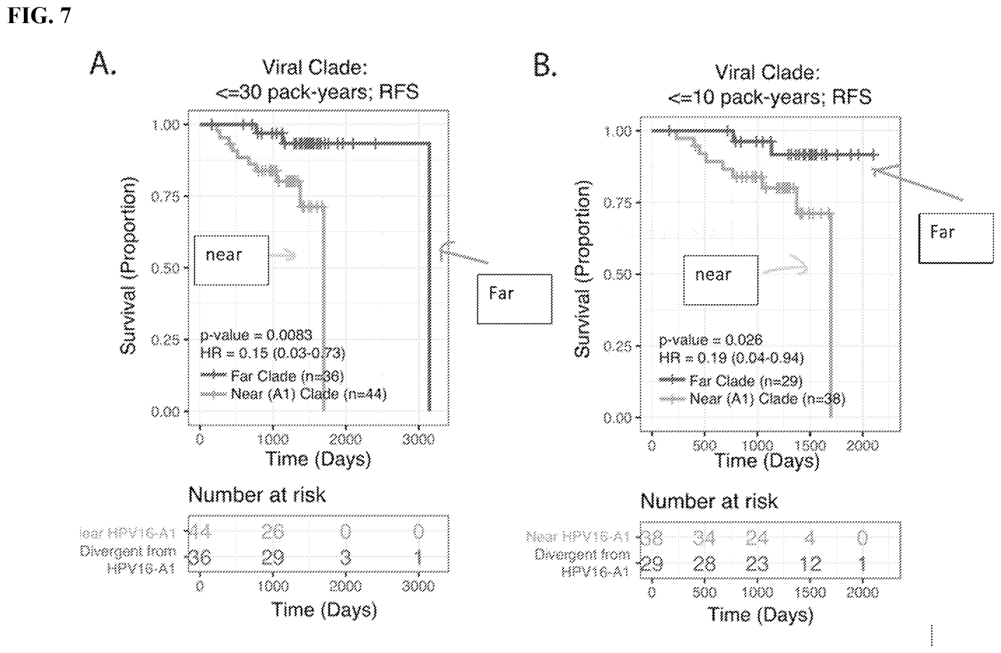

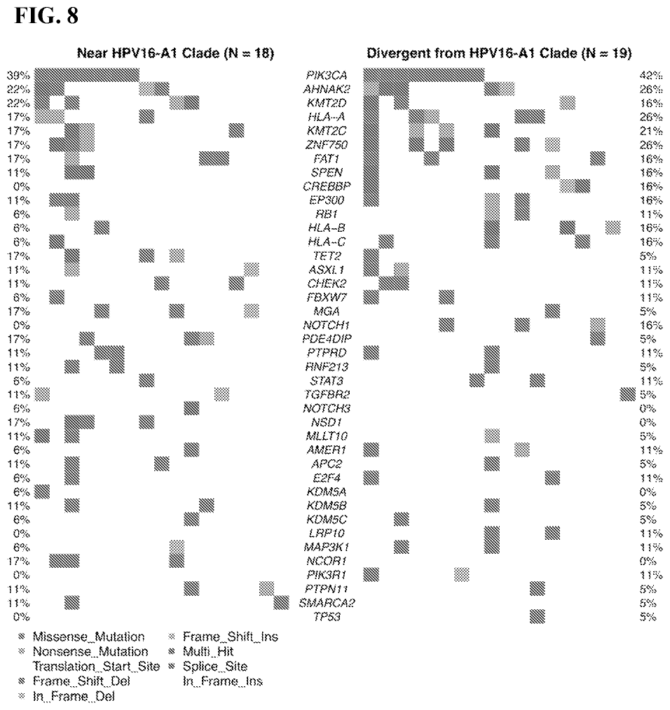

The maximum parsimony phylogeny of oncogenic oropharyngeal HPV16 viruses could be reasonably divided into two clades, with membership being highly correlated to the number of non-synonymous polymorphisms (as well as total number of SNPs) relative to the HPV16-A1 reference sequence (FIG. 4 panel A). These de-novo clades were also associated with variable recurrence-free survival. Consistent with the sub-lineage analysis, the clade more divergent from (far) HPV16-A1 demonstrated relatively favorable recurrence-free survival (FIG. 4 panel B). This association persisted in a multivariate regression model including viral clade (HR=0.16, 95% CI=0.04-0.61, p-value=0.007), early vs. advanced AJCC8 summary stage (HR=1.6, 95% CI=0.45-5.9, p-value=0.45), and smoking exposure greater than 10 pack-years (HR=1.6, 95% CI=0.61-4.4, p-value=0.33). Viral clade remained associated with RFS on subset analysis excluding patients with extensive (>30 pack-years) tobacco smoke exposure. The association with RFS also persisted when excluding all patients with greater than 10 pack-years of exposure (FIG. 7).Because clinical stage and tobacco exposure history are accepted clinical prognostic markers for HPV+OPSCC, their correlation was directly queried with viral clade, no association was detected (FIG. 4 panels C and D). As was found in the sub-lineage analysis, no difference in antigenicity was identified (FIG. 4 panel E). A subset of tumors (n=37) also had available matched normal DNA sequencing which allowed somatic variant calling (FIG. 8). Mutations in PIK3 (A have been reported as a poor prognosticator but did the frequency of these mutations was similar between the near and far clades (FIG. 8). One TP53 variant was identified in a patient with a low-risk, divergent clade HPV16 genotype. Similar to the above sub-lineage analysis, an increased number of genomic CNV was associated with the near A1 clade (p=0.015). Follow-up time, race, patient age, patient sex, treatment modality, and anatomic site of tumor origin were also examined, stratified according to HPV16 viral clade (Table 2). There was a trend towards more white patients in the near A1, high-risk clade. Follow up time was longer in the divergent (from HPV16-A1) low-risk clade.

Origins of Genomic Diversity in Oncogenic Oropharyngeal HPV16—Environmental Vs. Intra-Tumoral:

Considering that tumors harboring HPV16 genomes more distantly related to HPV16-A1 had distinct biological characteristics, it was considered whether the origins of the clonal (environmental) HPV genomic diversity of these groups were also distinct. SNPs and their related trinucleotide contexts were generated for identified non-synonymous polymorphisms (in the HPV16 genome). The analysis was limited to those polymorphisms identified in <25% of all tumors investigated (limiting analysis to even more uncommon polymorphisms did not change the qualitative results). Tumors groups were then stratified by viral clade as in FIG. 4, and non-negative matrix factorization for the COSMIC Signatures (V2)⁴⁷ was performed (FIG. 5 panels A and B). Diversity amongst HPV16 genomes more related to HPV16-A1 (FIG. 5 panel A) was dominated by Signature 9 (NMF weight of 0.61) which is thought to be related to DNA Polymerase Eta related mutagenesis. Alternatively, diversity amongst HPV16 genomes more distantly related to HPV16-A1 (FIG. 5 panel B) was dominated by Signature 3 (NMF weight of 0.50) which is thought to be related to defective homologous recombination repair of double-strand breaks. Although the origins of the investigated SNPs are unknown, the trinucleotide context-dependent base substitutions appear to be grossly dissimilar between these two groups of tumors bolstering the concept that they are biologically distinct groups of tumor viruses.

Sub-clonal viral polymorphisms, which likely arose during or after oncogenesis also demonstrated a distinct pattern of trinucleotide contexts, compared to clonal polymorphisms (FIG. 2 panel E) suggesting a prominent role for APOBEC mediated mutagenesis in defining viral subclones in HNSCC tumor cells. The number of distinct viral subclones were estimated by the Bayesian information criteria (BIC) after clustering of viral sub-clonal VAFs. 31% of tumors had discernable viral genomic sub-clonal populations with an allele fraction >5% (FIG. 5 panel C). Sub-clonal populations were often defined by SNPs (in addition to similar VAFs), had similar trinucleotide contexts, and were close in genomic space, suggestive of multi-mutational kataegis events, which may be the result of APOBEC mediated mutagenesis (FIG. 5 panel D). Neither the frequency of sub-clonal polymorphisms nor sub-clonal populations were associated with patient outcome by any metric investigated.

### Validation of HPV16 Genotyping by RNA Sequencing:

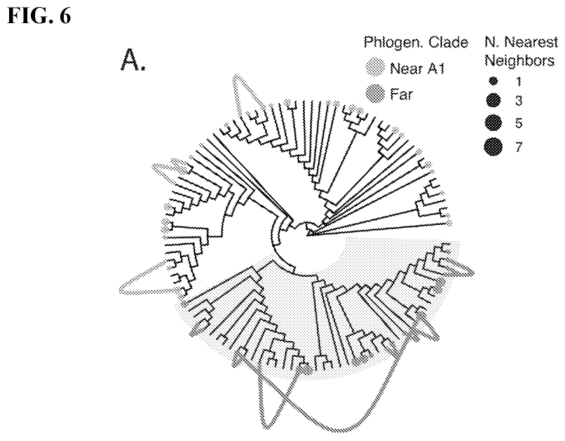

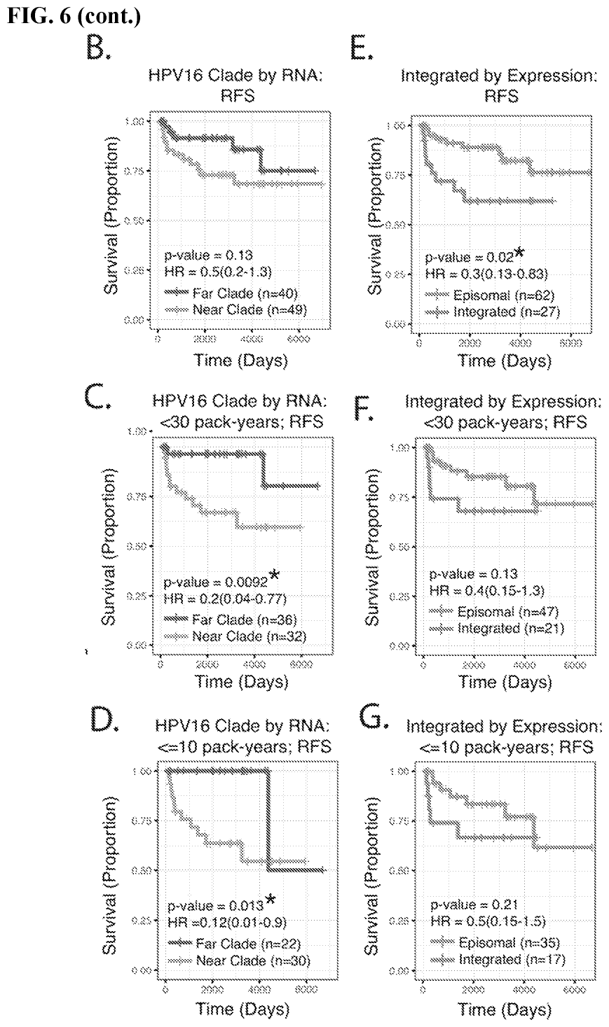

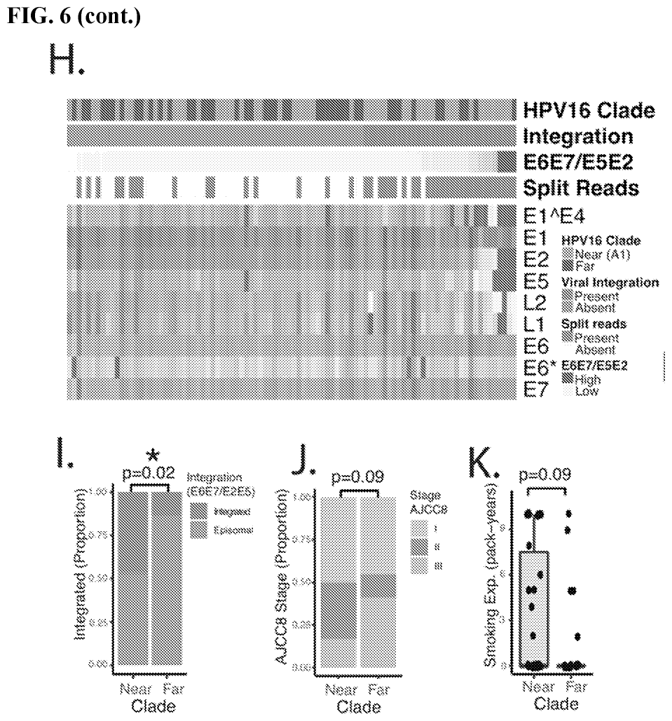

To determine if RNA sequencing data was sufficient to assign the HPV16 genotype of a given tumor to the viral clades investigated in FIG. 4, 13 patients from the DNA sequencing cohort were also subject to RNA sequencing. Gross agreement of SNPs as determined by DNA and RNA sequencing were seen in all 13 cases. Excluding areas with zero coverage in the RNA data, clade membership was assigned according to the nearest neighbors in sequence space (excluding the parent sample) based on JC69 sequence distance. In 100% (13/13) of cases the viral clade assigned by DNA sequencing was recovered by analysis of the RNA sequencing data. With a single exception, nearest neighbors assigned by RNA were quite close in phylogenetic space to the parent tumor based on DNA (FIG. 6 panel A). Based on prevalence of tumors in the two clade groups, of the probability of 13/13 correct classifications by random chance would be ˜3.8*10\hat{ }-5. Following these data confirming that RNA sequencing correctly classified HPV to near or far clades, further validation studies were performed using tumor with RNA sequencing data alone.

Validation RNA sequencing Cohort:

Considering the strong relationship of HPV16 viral genotype to recurrence free survival in the cohort presented above, studies were performed to validate the finding in a secondary, independent set of patients. Inclusion criteria were the same as the DNA sequencing cohort, with the exception that p16 status was not examined, because many patients with older archival tissue from OPSCC were not subject to routine p16 testing. 120 patients with archival FFPE from OPSCC were identified and processed for next generation RNA sequencing. Of these, 89 patients were found to express HPV16 genes and had sufficient clinical data for inclusion.

### Validation of HPV16 Genotype by RNA Sequencing as Predictor of RES:

Prior studies have demonstrated a relationship between viral genomic integration and survival in HPV+OPSCC. To compare the prognostic values of HPV16 genotype and viral integration, integration status was assigned using a combination of human-viral split read pair identification, as well as the ratio of expression of HPV16 genes E6/E7 to E5/E2 (FIG. 6 panel H). A trend towards improved RFSl for HPV16 genotypes divergent from the A1 clade was noted in the RNA-only cohort (FIG. 6 panel B). Patients with high degrees of tobacco smoke exposure were somewhat overrepresented in this new data set, with 20/89 (22%) patients having >=30 pack-years of tobacco smoke exposure. Subset analysis of patients with <30 pack years of tobacco smoke exposure revealed a strong relationship to RFS, with improved survival in patients with more divergent HPV16 sequences (FIG. 6 panel C). This relationship persisted in the subset of patients with <=10 pack-years of smoking exposure (FIG. 6 panel D). Consistent with prior reports, viral integration was prognostic of poor RFS when all patients were examined (FIG. 6 panel E) (Koneva et al. Mol Cancer Res MCR. 2018; 16(1):90-102). However, this association lost significance for patients with less than 30 pack-years or 10 pack-years of smoking exposure (FIG. 6 panels F and G). Within the subset of patients with <=10 pack-years of smoking exposure, HPV16 genotypes closely related to HPV16-A1 were more likely to be integrated (p=0.02, Chi-squared test) (FIG. 6 panel I). No significant differences in AJCC8 stage or tobacco smoke exposure were noted in the <=10 pack-year sub-group, but the far clade (favorable prognosis group) trended toward higher stage and fewer pack years (p=0.09) (FIG. 6 panels J and K).

In summary, two independent cohorts of HPV16+ OPSCC tumors were analyzed for a total of 187 cases, representing one of the largest reported series of HPV+ OPSCC with viral sequencing. Since HPV16 accounts for greater than 90% of OPSCC, and HPV sub-type may correlate with outcome (Nichols et al. J Otolaryngol—Head Neck Surg. 2013; 42(1):9), we limited our study to HPV16 positive tumors as confirmed by DNA or RNA sequencing. This study has uncovered a surprising degree of genomic diversity in oncogenic HPV16 viral genomes, with 93 distinct protein coding HPV16 genomes amongst the 104 tumors examined. This rich diversity has been previously under investigated in OPSCC.

HPV+ and HPV-negative OPSCC respond quite differently to treatment, with much better survival for patients with HPV+tumors (Ben-David et al. Nature. 2018; 560(7718):325-330). Excellent rates of 5-year disease control often come at the cost of relatively high rates of unfortunate treatment-related morbidities such as gastrostomy tube dependence and osteoradionecrosis of the mandible. Although there is interest in de-intensification of therapy for HPV+ OPSCC (Cheraghlou et al. Cancer. 2018; 124(4):717-726; Chera et al. Cancer. 2018; 124(11):2347-2354), inaccuracy of current prognostic markers such as smoking history, stage or radiologic characteristics in predicting long-term disease control are of concern to clinicians who fear undertreating a patient who may otherwise be cured with standard regimes (Cheraghlou et al. Cancer. 2018; 124(4):717-726). However, long-term analysis of patients treated with high-intensity chemoradiation protocols has revealed increased non-cancer mortality that may negate the survival advantage of aggressive therapy (Forastiere et al. J Clin Oncol Off J Am Soc Clin Oncol. 2013; 31(7):845-852; Tasoulas et al. Cancer Med. 2021; 10(10):3231-3239).

This study demonstrates that targeted HPV16 sequencing or total RNA sequencing from FFPE from routine clinical specimens is sufficient to acquire clinically relevant and risk-stratifying information that could inform treatment decisions. The prognostic value of HPV16 genotype classification demonstrated herein within two independent cohorts for the subgroup of patients with no more than 10 pack-years of tobacco smoke exposure (the current eligibility threshold for reduced treatment intensity at our center and others). Although sequencing of the relatively small ˜7900 base HPV genome from FFPE would be easily performed in many modern clinical laboratories, circulating cell-free HPV DNA shed from OPSCC cancer cells can also be detected, quantified and genotyped from clinical blood draw specimens. Therefore, it is also may be possible to similarly acquire prognostic HPV16 genotypic data from clinical blood samples; making HPV16 genotyping a relatively ideal biomarker for clinical application.

We have previously reported that genomic instability determined by copy number variant burden is also prognostic in HPV16+ OPSCC (Schrank et al. 2021 Cancer 127(15):2788-2800; incorporated herein by reference in its entirety). In the present study it is shown that HPV16-A1 and closely related genotypes are also associated with increased CNV burden. Genomic CNVs identified were primarily numerical chromosomal aberrations or large-scale sub-chromosomal amplifications or losses. This correlation could be explained by the fact that high-risk HPV16 infection and expression of HPV16 E6 and E7 have been demonstrated to directly result in numerical and structural chromosomal instability (Duensing and Münger. Cancer Res. 2002; 62(23):7075-7082).

Studies have established that HPV infection both induces replication stress, causes DNA damage, disrupts cellular DNA double-strand break (DSB) repair via multiple mechanisms, and that HPV-positive head and neck cancer cells are deficient in homologous recombination repair of double-strand breaks. Double-strand break repair dysfunction has been broadly linked to genomic instability in human cancers. Therefore, it is possible that functional polymorphisms in the HPV16 genome result in differential modulation of the host of human DDR factors known to be influenced by high risk HPV infection. Low-risk HPVs (HPV6, HPV11) have been reported to have relatively diminished ability to disrupt the human DNA damage response (DDR) (Wallace N A. Trends Microbiol. 2020; 28(3):191-201).

Although somewhat counterintuitive that double-strand break mediated viral mutagenesis was correlated with the low-risk group of tumors (divergent from A1) with relatively stable human genomes (FIG. 5 panel B). This may reflect relatively ineffectual recruitment of human DDR factors to viral replications centers rather than heightened disruption of the DDR in general. Indeed, experimental depletion of host factors involved in the replication of the HPV genome has been demonstrated to primarily decrease the fidelity (increased viral mutagenesis) without greatly altering the throughput of the replicative process. Tumors with HPV16 genomes more similar to the A1 sub-lineage were associated with Polymerase Eta (POLH)-related variants. It is also interesting that E1\hat{ }E4, E2, and to some degree E5—the HPV proteins known to be involved in HPV replication, HPV gene expression, and recently shown to drive alternative episome-based carcinogenesis—were found to be the least conserved amongst HPV16 viral genes (FIG. 2 panels A-B).

Prior studies of uterine cervical cancer have demonstrated that HPV18 is more likely to integrated as compared to HPV16, suggesting that HPV genotype can influence the likelihood of genomic integration during carcinogenesis (McBride & Warburton. PLOS Pathog. 2017; 13(4): e1006211). In the present work, the RNA sequencing cohort which allowed viral integration analysis, demonstrated a relationship between viral integration and HPV16 genotype in patients with <=10 pack-years of smoking. These data suggest that even more subtle genotypic variations in the HPV genome (amongst HPV16 viruses) may influence the chance of genomic integration. The data herein from two independent patient cohorts suggests that molecular risk-stratification based on HPV16 genotype may be used as a genomic tool for the identification of very low-risk patients which may enable safe treatment de-escalation and limit long-term, treatment-related toxicity which is a key goal in the field.

DNA sequencing data were collected as a part of the UNCseq tumor sequencing program. The UNCseq targeted sequencing platform involves sequencing exons of a custom list of 650 human genes (covering 3.4 M bases) and 10 pathogen genome segments in fixed or frozen cancer tissue and matched germline DNA from consenting local patients. This custom sequencing platform provided targeted coverage of all HPV16 open reading frames. Tumor sample identifiers and genomic data were derived from the clinical trial LCCC1108: Development of a Tumor Molecular Analyses Program and Its Use to Support Treatment Decisions. This IRB-approved trial opened in 2011. All studies were done with the approval of the Institutional Review Board, patient participation required written informed consent, and all studies were conducted in accordance with recognized ethical guidelines as described in U.S Common Rule. The UNCseq database was queried for all patients with HPV+ oropharyngeal cancer.

Via chart review of electronic medical records, demographic information was obtained for each study subject, including age, gender, race, and smoking history. Clinical stage at presentation according to the AJCC staging system (AJCC 8^{th} edition) was recorded, clinical AJCC8 staging was used, considering that many patients did not receive surgical treatment. Recurrence-free survival was defined from the date of initial diagnosis to the date at which evidence of recurrent disease was first documented after primary treatment. Cases that presented with distant metastasis (n=2) were excluded from recurrence free survival analysis. All survival analysis was performed with the R Survival package. Cox proportional hazards models were implemented with the coxph function.

### DNA Isolation, Library Preparation, and Sequencing:

A pathologist examined H&E-stained slides from each case to confirm the diagnosis of squamous cell carcinoma. Automated DNA extraction was from FFPE tissue sections using the Promega Maxwell MD×16™ instruments (Promega) and then fragmented by sonication. Subsequent quality assessments were performed by ultraviolet absorbance and quantity assessments. During

DNA isolation and library preparation, DNA concentration was measured by fluorometry and DNA quality was evaluated using the Agilent 2100 Bioanalyzer high sensitivity assay. DNA libraries were pooled for deep sequencing using an Illumina HiSeq2500™ sequencer. Excluding the hypervariable region 3150-3351, median coverage of the HPV16 genome was 7904 with an IQR of 6867-7953. The hypervariable region 3150-3351 had median coverage of 3307 with an IQR of 837-7377. Data have been made publicly available through dbGaP accession number phs001713.v1.p1.

### RNA Isolation, Library Preparation, and Sequencing:

Formalin-fixed paraffin-embedded (FFPE) tissue samples were prepared for RNA isolation using the Maxwell 16 MDx Instrument (Promega AS3000) and the Maxwell 16 LEV RNA FFPE Kit (Promega AS1260) following the manufacturer's protocol (Promega 9FB167). After a pathology review of a hematoxylin and eosin (H&E) stained slide to identify tumor area, RNA was extracted from unstained slides using macrodissection. Total RNA quality was measured using a NanoDrop spectrophotometer (Thermo Scientific ND-2000C) and a TapeStation 4200 (Agilent G2991AA). Total RNA concentration was quantified using a Qubit 3.0 fluorometer (Life Technologies Q33216). Libraries were prepared with Illumina TruSeq Stranded Total RNA with Ribo-Zero protocol. Libraries were sequenced on an Illumina HiSeq2500 sequencer. Paired end read data, with read lengths of 75 were collected.

### Bioinformatics:

Raw reads were aligned to the human genome plus a comprehensive library of HPV virus sequences, using compiled reference sequences from the ViFi analysis pipeline. Human genomic mutations were not investigated as only a subset of samples had matched normal DNA sequencing. Tumors were considered to be HPV16 positive if they had more than 20,000 reads mapping to the HPV16 genome. The HPV16 A1 genotype, RefSeq NC_001526.4 (incorporated herein by reference in its entirety) was selected as the primary reference sequence for this study. The Varscan pipeline was used for variant/polymorphism calling, using the reference-free approach. The SnpEff pipeline was used to assign and prioritize variant effects. Consensus sequences were derived from the variant calls which represented clonal variants using in-house scrips. SNPs were only analyzed if the sequencing depth was >=100 and VAF >=0.5. Variants were considered subclonal if VAF <=0.9. The HPV tumor genomes were then aligned against each other and 16 reference HPV16 sub-lineage sequences. Alignments were imported into R for phylogenetic analyses, where maximum parsimony phylogeny was constructed using default parameters provided in the phangorn package. To assign the nearest sub-lineages to the tumor HPV genomes, the R phangorn package was used to construct a sequence distance matrix for each tumor HPV and 16 reference sequences. Each tumor sample was assigned to an HPV16 sub-lineage based on the “closest” reference sequence using the “JC69” distance.

### Neo-Antigenicity Analysis—

For each patient, targeted genomic sequencing including HLA-A, HLA-B, HLA-C was processed by the Optitype pipeline. Comparison with matched normal (blood) sequencing data, where available, demonstrated remarkable consistency of the results between tumor and matched normal blood. Therefore, tumor sequencing data was utilized for HLA typing for the purposes of this study. The netMHCpan pipeline was then applied to generate predicted binding affinities of all possible patient-matched viral peptide and MHC pairs. An empiric threshold of <325 nM was applied to identify high affinity interaction between MHC and viral peptides. The log fraction of high-affinity to all possible viral peptide MCH interactions was also investigated as a neo-antigenicity metric, as homozygous haplotypes of HLA loci in some patients made the background number of potential peptide-HLA interactions variable between patients.

### APOBEC and Mutational Signature Analysis—

Based on prior reports, mutational contexts represented by COSMIC Version 2 Signature 13 (C>G variants) and Signature 2 (C>T variants) were considered to be potentially APOBEC related (Revathidevi et al. Cancer Lett. 2021; 496:104-116). Using the trinucleotide contexts most prominent in COSMIC Signatures 2 (T [C>T] A, T [C>T] C, T [C>T] T) and 13 (T [C>G] A, T [C>G] C, T [C>G] T), alterations were counted as potentially APOBEC related. Chi-squared test was applied to determine differences in minor proportions of ABOBEC related variants. Nonnegative matrix factorization was used to estimate the contribution of different mutational process influencing the HPV16 genome. This analysis was implemented and visualized with the R package DeconstructSigs, after generating trinucleotide context SNP matrices with in-house scripts.

### Somatic Variant Calling—

Sequencing data were routed through an automated pipeline including a somatic workflow using paired tumor and normal libraries to detect somatic mutations, large and small indels, structural variants, and pathogenic organisms. Raw sequences were aligned using the BWA-mem algorithm and refined using an assembly based realignment process to allow for accurate alignment of complex sequence variation. Only high confidence variants with phred-scaled quality scores greater than 30 were included in the analysis. Average target coverage was ˜1000x.

### Copy Number Variant Calling—

Copy number calls were generated with the SynthEx algorithm using the tumor sequencing data and a library of 200 un-matched normal samples sequenced with the same technique. The SynthEx pipeline utilizes both on and off target reads to allow large-segment copy number variant calling across the human genome. A conservative approach was taken. Thirty replicates varying the parameter k (number of nearest neighbor) were done per tumor and the model with the fewest deviations from the expected copy number of 2 was selected. Sex chromosomes were excluded.

### RNA Data Quantification—

The HPV16 A1 genotype, RefSeq NC_001526.4 was selected as the primary reference sequence for this study. Salmon was used to quantify RNA reads for NC_001526.4 as well as hg38 (Patro et al. Nat Methods. 2017; 14(4):417-419). Viral transcripts read counts were transformed into log 2 viral read counts per million total mapped reads (human and viral) prior to visualization and analysis. Tumors were considered to be HPV16 positive if there were more than 2000 reads mapping to HPV16 and the log 10 ratio of HPV16/Human reads was >−4.5. 24 cases were excluded based on these criteria.

### Viral Integration Analysis—

The ViFi pipeline was used to identified discordant (human—viral) read pairs which cluster to potential integration sites in the human and HPV genomes. Tumors with more than 25 clustered discordant read pairs were classified as positive for (discordant) split reads. The ratio HPV16 E6 and E7 to HPV16 E5 and E2 was calculated for each tumor based on quantification from Salmon. Above a ratio of −0.304, 88% of tumors also displayed locus specific clusters of human-viral split read pairs. Below this threshold only 26% of tumors had human-viral split read pairs. Therefore, tumors with E6E7/E5E2 ratio above this threshold were considered to have an integrated pattern of viral gene expression.

### Viral Genotyping by RNA—

First partial consensus sequences were constructed from viral BAM files output from the ViFi pipeline. With the goal getting an approximate sequence, the Varscan pipeline was used for variant/polymorphism calling, using the reference-free approach, and lax parameters with minimal coverage requirement of 1, VAF minimum of 0.51 and no p-value cut off. These “variants” were used to construct approximate consensus sequences in area of the viral genome covered by the sequencing data. See Supplemental FIG. 3, for an illustration of the gross quality of HPV16 genotype data as ascertained by RNAseq. JC69 sequence distances from these partial, approximate viral sequences were then calculated using the R phangorn package. Tumors were classified into viral clades based on the majority voting of the three nearest neighbors from the DNA sequencing cohort. For the 13 tumors with both DNA and RNA sequencing, this method recovered the viral clade in 100% of cases (FIG. 6 panel A).

### Inclusion Criteria:

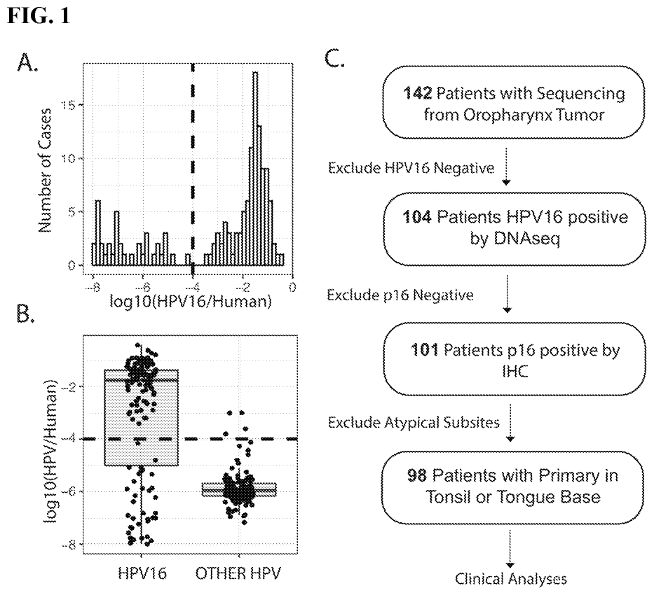

The UNCseq database was queried for p16+ tumors originating from the anatomic oropharynx (tonsil or tongue base) with available tumor sequencing data as well as data on stage, treatment strategy, clinical outcome, and histopathology available. HPV16 positivity was confirmed by DNA sequencing reads which mapped to the HPV16 genome. For the DNA sequencing cohort, patients were excluded from clinical analyses if tumors were not p16+ or were from atypical oropharyngeal sub-sites (midline soft palate or lateral oropharyngeal wall) (FIG. 1). The confirmatory RNA sequencing cohort included cases dating prior to routine p16 IHC was performed at our institution, therefore, p16 status was not considered in this secondary cohort.

### Example 2: HPV16-A1 Genotype is Associated with Poor Recurrence-Free Survival in HPV16-Associated Squamous Cell Carcinoma of the Oropharynx

HPV-positive squamous cell carcinoma of the oropharynx (HPV+ OPSCC) is the most prevalent HPV-associated malignancy in the United States and is primarily caused by HPV16. Favorable treatment outcomes have led to increasing interest in treatment de-escalation to reduce treatment-related morbidity. Prognostic biomarkers are needed to identify appropriately low-risk patients for reduced treatment intensity. Large series of complete HPV16 genome sequencing from HPV+ OPSCC tumors are lacking in the literature. Therefore, this study tested the hypothesis that HPV16 genotype is prognostic of recurrence-free survival (RFS) in HPV16+ OPSCC.

Materials/Methods: Targeted sequencing of 104 patients with HPV16+ OPSCC tumors was performed, providing complete coverage of all HPV16 open reading frames. Clinical features were retrospectively extracted from the medical record. A second cohort of OPSCC patients was sequenced using total RNA sequencing, which identified 89 patients with HPV16+ OPSCC for analysis.

Results: A high degree of coding diversity in the HPV16 was identified, with 93 distinct protein-coding HPV16 genotypes amongst the 104 patients subject to HPV (DNA) sequencing. As found in uterine cervical carcinoma, E7 was the most conserved amongst HPV16 viral genes. Sub-clonal variants were more likely to be non-synonymous and were enhanced for APOBEC-related mutagenesis. The HPV16-A1 sub-lineage was the most prevalent (approximately 70%). Genotypes closely related to HPV16-A1 were associated with increased numbers of copy-number variants in the human genome. Genotypes divergent from HPV16-A1 were strongly associated with favorable RFS as compared to HPV16-A1 (or similar genotypes); this finding was independent of tobacco smoke exposure. HPV16 genotypes divergent from HPV16-A1 were subsequently validated in an independent cohort (subject to RNA sequencing), to be associated with improved RFS in patients with moderate (less than 30 pack-years) and low (no more than 10 pack-years) of tobacco smoke exposure.

Conclusion: HPV16 viral genotype is highly diverse in HPV associated OPSCC. Sequence divergence from the HPV16-A1 reference sequence is strongly associated with improved RFS in patients with moderate to no tobacco smoke exposure. This finding was confirmed in two independent cohorts. HPV16 genotype is a promising biomarker to guide therapeutic decision-making related to de-escalation therapy. Prognostic genotypic information can be obtained from clinical samples stored in FFPE applying either DNA or RNA sequencing technology.

### Tables

TABLE 1
Clinical Features Stratified by HPV16 Sub-lineage: DNA Sequencing
Cohort. P-values - represent Chi-squared test applied to categorical
data and t-test for continuous numerical data.

| | HPV16-A1 | HPV16-Other | |
|---|---|---|---|
| | n = 70 | n = 28 | p-value |
| Age (mean(SD)) | 57.90 (9.70) | 58.39 (10.28) | 0.824 |
| Sex (% Female) | 12 (17.1) | 2 (7.1) | 0.338 |
| Race (% White) | 66 (94.3) | 22 (78.6) | 0.051 |
| AJCC8 Stage | | | 0.785 |
| I | 42 (60.0) | 18 (64.3) | |
| II | 18 (25.7) | 5 (17.9) | |
| III | 9 (12.9) | 4 (14.3) | |
| IV | 1 (1.4) | 1 (3.6) | |
| Smoking pack-years | 11.32 (18.87) | 11.26 (20.83) | 0.989 |
| (mean(SD)) | | | |
| Follow-up-time in days | 1247.01 (493.20) | 1489.57 (697.29) | 0.055 |
| (mean(SD)) | | | |
| Treatment Strategy (%) | | | 0.742 |
| chemoradiation | 52 (74.3) | 17 (60.7) | |
| palliative chemo | 1 (1.4) | 1 (3.6) | |
| radiation | 2 (2.9) | 1 (3.6) | |
| surgery alone | 3 (4.3) | 2 (7.1) | |
| surgery with adjuvant | 12 (17.1) | 7 (25.0) | |
| Anatomic Subsite | 38 (54.3) | 18 (64.3) | 0.498 |
| (% Tonsil) | | | |

TABLE 2
Clinical Features Stratified by HPV16 Viral Clade: DNA Sequencing Cohort.
Clades as assigned in FIG. 4. P-values - represent Chi-squared test
applied to categorical data and t-test for continuous numerical data.

| | Near HPV16-A1 | Divergent from A1 | |
|---|---|---|---|
| | n = 56 | n = 42 | p-value |
| Age (mean(SD)) | 58.41 (9.77) | 57.55 (9.99) | 0.669 |
| Sex (% Female) | 9 (16.1) | 5 (11.9) | 0.771 |
| Race (% White) | 53 (94.6) | 35 (83.3) | 0.135 |
| AJCC8 Stage | | | 0.987 |
| I | 35 (62.5) | 25 (59.5) | |
| II | 13 (23.2) | 10 (23.8) | |
| III | 7 (12.5) | 6 (14.3) | |
| IV | 1 (1.8) | 1 (2.4) | |
| Smoking pack-years | 11.38 (18.80) | 11.20 (20.27) | 0.964 |
| (mean(SD)) | | | |
| Follow-up-time in days | 1208.50 (474.81) | 1460.07 (647.13) | 0.029 |
| (mean(SD)) | | | |
| Treatment Strategy (%) | | | 0.901 |
| chemoradiation | 41 (73.2) | 28 (66.7) | |
| palliative chemo | 1 (1.8) | 1 (2.4) | |
| radiation | 2 (3.6) | 1 (2.4) | |
| surgery alone | 3 (5.4) | 2 (4.8) | |
| surgery with adjuvant | 9 (16.1) | 10 (23.8) | |
| Anatomic Subsite | 30 (53.6) | 26 (61.9) | 0.536 |
| (% Tonsil) | | | |

TABLE 3
Clinical Features Stratified by HPV16 Viral Clade: RNA Sequencing Validation
Cohort. Clades as assigned in FIG. 6 panel A. P-values - represent Chi-squared
test applied to categorical data and t-test for continuous numerical data.

| | Near HPV16-A1 | Divergent from A1 | |
|---|---|---|---|
| | n = 49 | n = 40 | p-value |
| Age (mean(SD)) | 56.56 (8.98) | 56.17 (7.78) | 0.83 |
| Sex (% Female) | 5 (10.2) | 1 (2.5) | 0.31 |
| Race (% White) | 46 (93.9) | 36 (90.0) | 0.779 |
| AJCC8 Stage | | | 0.025 |
| I | 27 (56.2) | 15 (37.5) | |
| II | 14 (29.2) | 9 (22.5) | |
| III | 7 (14.6) | 16 (40.0) | |
| IV | 0 (0) | 0 (0) | |
| Smoking pack-years | 15.57 (18.22) | 15.00 (19.34) | 0.886 |
| (mean(SD)) | | | |
| Follow-up-time in days | 2692.29 (1703.43) | 2624.00 (1762.68) | 0.853 |
| (mean(SD)) | | | |
| Treatment Strategy (%) | | | 0.73 |
| chemoradiation | 45 (91.8) | 36 (90.0) | |
| palliative chemo | 0 (0) | 0 (0) | |
| radiation | 2 (4.1) | 1 (2.5) | |
| surgery alone | 0 (0) | 0 (0) | |
| surgery with adjuvant | 2 (4.1) | 3 (7.5) | |
| Anatomic Subsite | 29 (59.2) | 17 (42.5) | 0.31 |
| (% Tonsil) | | | |

### Sequences

The following samples (determined by phylogenetic analysis) are defined as low risk/de-escalation group:

“UNCseq1628” SEQ ID NO: 13, “UNCseq1770” SEQ ID NO:23, “UNCseq2162” SEQ ID NO: 58, “UNCseq1750” SEQ ID NO:22, “UNCseq1864” SEQ ID NO:30, “UNCseq1867” SEQ ID NO:31, “UNCseq2234” SEQ ID NO:62, “UNCseq2491” SEQ ID NO: 80, “UNCseq2539” SEQ ID NO:82, “UNCseq2012” SEQ ID NO:48, “UNCseq2554” SEQ ID NO:83, “UNCseq2771” SEQ ID NO:93, “UNCseq1796” SEQ ID NO:25, “UNCseq0733” SEQ ID NO: 1, “UNCseq2051” SEQ ID NO:52, “UNCseq2789” SEQ ID NO: 102, “UNCseq2106” SEQ ID NO:56, “UNCseq1742” SEQ ID NO:20, “UNCseq1140” SEQ ID NO:4, “UNCseq1849” SEQ ID NO:28, “UNCseq 1980” SEQ ID NO:41, “UNCseq2005” SEQ ID NO:45, “UNCseq2105” SEQ ID NO:55, “UNCseq1360” SEQ ID NO: 6, “UNCseq1525” SEQ ID NO:7, “UNCseq1891” SEQ ID NO:32, “UNCseq 1662” SEQ ID NO: 14, “UNCseq 1924” SEQ ID NO:36, “UNCseq2292” SEQ ID NO:66, “UNCseq1994” SEQ ID NO:43, “UNCseq2007” SEQ ID NO:46, “UNCseq1834” SEQ ID NO:26, “UNCseq2783” SEQ ID NO: 100, “UNCseq2786” SEQ ID NO: 101, “UNCseq1693” SEQ ID NO: 66, “UNCseq2033” SEQ ID NO:51, “UNCseq0848” SEQ ID NO:2, “UNCseq2249” SEQ ID NO: 63, “UNCseq2794” SEQ ID NO: 106, “UNCseq2393” SEQ ID NO:72, “UNCseq2576” SEQ ID NO:84, “UNCseq2795” SEQ ID NO: 107, “UNCseq1697” SEQ ID NO: 17, “UNCseq1938” SEQ ID NO:38, “UNCseq1991” SEQ ID NO:42

Samples recited below which are not named above are defined as high risk/do not de-escalate.

UNCseq0733 (SEQ ID NO:1); UNCseq0848 (SEQ ID NO:2); UNCseq1009 (SEQ ID NO:3); UNCseq1140 (SEQ ID NO:4); UNCseq1310 (SEQ ID NO:5); UNCseq1360 (SEQ ID NO:6) UNCseq1525 (SEQ ID NO:7); UNCseq1527 (SEQ ID NO:8); UNCseq1583 (SEQ ID NO:9); UNCseq 1588 (SEQ ID NO:10); UNCseq1593 (SEQ ID NO:11); UNCseq1610 (SEQ ID NO: 12); UNCseq1628 (SEQ ID NO:13); UNCseq1662 (SEQ ID NO:14); UNCseq1678 (SEQ ID NO:15); UNCseq1693 (SEQ ID NO:16); UNCseq1697 (SEQ ID NO:17); UNCseq1710 (SEQ ID NO:18); UNCseq1712 (SEQ ID NO:19); UNCseq 1742 (SEQ ID NO: 20); UNCseq1743 (SEQ ID NO:21); UNCseq1750 (SEQ ID NO:22); UNCseq1770 (SEQ ID NO:23); UNCseq1787 (SEQ ID NO:24); UNCseq1796 (SEQ ID NO:25); UNCseq1834 (SEQ ID NO:26); UNCseq1840 (SEQ ID NO:27); UNCseq1849 (SEQ ID NO: 28); UNCseq1851 (SEQ ID NO:29); UNCseq1864 (SEQ ID NO:30); UNCseq1867 (SEQ ID NO:31); UNCseq1891 (SEQ ID NO:32); UNCseq1897 (SEQ ID NO:33); UNCseq1906 (SEQ ID NO:34); UNCseq1918 (SEQ ID NO:35); UNCseq1924 (SEQ ID NO: 36); UNCseq1930 (SEQ ID NO:37); UNCseq1938 (SEQ ID NO:38); UNCseq1954 (SEQ ID NO:39); UNCseq1958 (SEQ ID NO:40); UNCseq1980 (SEQ ID NO:41); UNCseq1991 (SEQ ID NO:42); UNCseq1994 (SEQ ID NO:43); UNCseq2000 (SEQ ID NO: 44); UNCseq2005 (SEQ ID NO:45); UNCseq2007 (SEQ ID NO:46); UNCseq2010 (SEQ ID NO:47); UNCseq2012 (SEQ ID NO:48); UNCseq2025 (SEQ ID NO:49); UNCseq2032 (SEQ ID NO:50); UNCseq2033 (SEQ ID NO:51); UNCseq2051 (SEQ ID NO: 52); UNCseq2056 (SEQ ID NO:53); UNCseq2083 (SEQ ID NO:54); UNCseq2105 (SEQ ID NO:55); UNCseq2106 (SEQ ID NO:56); UNCseq2116 (SEQ ID NO:57); UNCseq2162 (SEQ ID NO:58); UNCseq2166 (SEQ ID NO:59); UNCseq2182 (SEQ ID NO: 60); UNCseq2209 (SEQ ID NO:61); UNCseq2234 (SEQ ID NO:62); UNCseq2249 (SEQ ID NO:63); UNCseq2253 (SEQ ID NO:64); UNCseq2254 (SEQ ID NO:65); UNCseq2292 (SEQ ID NO:66); UNCseq2298 (SEQ ID NO:67); UNCseq2333 (SEQ ID NO: 68); UNCseq2337 (SEQ ID NO:69); UNCseq2344 (SEQ ID NO:70); UNCseq2376 (SEQ ID NO:71); UNCseq2393 (SEQ ID NO:72); UNCseq2413 (SEQ ID NO:73); UNCseq2426 (SEQ ID NO:74); UNCseq2427 (SEQ ID NO:75); UNCseq2430 (SEQ ID NO: 76); UNCseq2450 (SEQ ID NO:77); UNCseq2468 (SEQ ID NO:78); UNCseq2476 (SEQ ID NO:79); UNCseq2491 (SEQ ID NO:80); UNCseq2523 (SEQ ID NO:81); UNCseq2539 (SEQ ID NO:82); UNCseq2554 (SEQ ID NO:83); UNCseq2576 (SEQ ID NO: 84); UNCseq2594 (SEQ ID NO:85); UNCseq2601 (SEQ ID NO:86); UNCseq2708 (SEQ ID NO:87); UNCseq2750 (SEQ ID NO:88); UNCseq2767 (SEQ ID NO:89); UNCseq2768 (SEQ ID NO:90); UNCseq2769 (SEQ ID NO:91); UNCseq2770 (SEQ ID NO: 92); UNCseq2771 (SEQ ID NO:93); UNCseq2772 (SEQ ID NO:94); UNCseq2773 (SEQ ID NO:95); UNCseq2774 (SEQ ID NO:96); UNCseq2775 (SEQ ID NO:97); UNCseq2778 (SEQ ID NO:98); UNCseq2779 (SEQ ID NO:99); UNCseq2783 (SEQ ID NO: 100); UNCseq2786 (SEQ ID NO:101); UNCseq2789 (SEQ ID NO:102); UNCseq2790 (SEQ ID NO:103); UNCseq2791 (SEQ ID NO:104); UNCseq2792 (SEQ ID NO:105); UNCseq2794 (SEQ ID NO:106); UNCseq2795 (SEQ ID NO:107)

Sample HPV16 sub-lineage genotypes.

A1 (SEQ ID NO:108); A2 (SEQ ID NO: 109); A3 (SEQ ID NO:123); A4 (SEQ ID NO:110); B1 (SEQ ID NO:111); B2 (SEQ ID NO:112); B3 (SEQ ID NO:113); B4 (SEQ ID NO:114); C1 (SEQ ID NO:115); C2 (SEQ ID NO:116); C3 (SEQ ID NO:117); C4 (SEQ ID NO:118); DI (SEQ ID NO:119); D2 (SEQ ID NO:120); D3 (SEQ ID NO:121); D4 (SEQ ID NO:122)

The foregoing is illustrative of the present invention, and is not to be construed as limiting thereof. The invention is defined by the following claims, with equivalents of the claims to be included therein.

1. A method of determining a treatment regimen for a subject having human papillomavirus (HPV) positive (HPV+) throat cancer and/or cervical cancer or a subject at risk for or suspected to have or develop HPV+ cancer and treating said subject with de-escalated treatment to the cancer, comprising:
a) obtaining a sample from the subject;
b) detecting a level of expression of HPV virus genome and/or genome product in the sample;
c) obtaining sequence information of the HPV viral genome and/or genome product in the sample;
d) comparing the sequence information obtained in c) to a reference HPV viral genome;
e) determining the prognosis of the subject upon treatment to the cancer, wherein a low divergence of the sequence information of the detected HPV virus genome and/or genome product as compared to the reference HPV viral genome identifies the subject as a candidate for standard treatment for the cancer, and wherein a greater divergence identifies the subject as a suitable candidate for de-escalated treatment for the cancer; and
f) treating the subject identified as a suitable candidate for de-escalated treatment, with de-escalated treatment to the cancer.

2-4. (canceled)

5. A method of de-escalating treatment of human papillomavirus (HPV) positive cancer in a subject having human papillomavirus (HPV) positive (HPV+) throat cancer and/or cervical cancer or a subject at risk for or suspected to have or develop HPV+ throat cancer and/or cervical cancer and undergoing standard treatment of the cancer, comprising:
a) obtaining a sample from the subject;
b) detecting a level of expression of HPV virus genome and/or genome product in the sample;
c) obtaining sequence information of the HPV viral genome and/or genome product in the sample;
d) comparing the sequence information obtained in c) to a reference HPV viral genome;
e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated treatment to the cancer, and wherein a greater divergence identifies the subject as a candidate for de-escalated categorizes the subject as having reduced risk of poor prognosis from de-escalated treatment to the cancer; and
f) treating the subject identified as having reduced risk of poor prognosis from de-escalated treatment with de-escalated treatment as compared to standard treatment for the cancer.

6. A method of treating human papillomavirus (HPV) positive (HPV+) cancer in a subject having human papillomavirus (HPV) positive (HPV+) throat cancer and/or cervical cancer or a subject at risk for or suspected to have or develop HPV+ throat cancer and/or cervical cancer, comprising:
a) obtaining a sample from the subject;
b) detecting a level of expression of HPV virus genome and/or genome product in the sample;
c) obtaining sequence information of the HPV viral genome and/or genome product in the sample;
d) comparing the sequence information obtained in c) to a reference HPV viral genome;
e) identifying the risk of poor prognosis of the subject from de-escalated treatment to the cancer, wherein a low divergence of the sequence information of the detected HPV virus genome and/or genome product as compared to the reference HPV viral genome categorizes the subject as having elevated risk of poor prognosis from de-escalated treatment to the cancer, and wherein a greater divergence categorizes the subject as having reduced risk of poor prognosis from de-escalated treatment to the cancer; and
f) treating the subject identified as having reduced risk of poor prognosis from de-escalated treatment with de-escalated treatment as compared to standard treatment for the cancer.

7. The method of claim 6, wherein obtaining sequence information of the HPV viral genome and/or genome product in the sample comprises obtaining RNA and/or DNA sequence information.

8. The method of claim 6, wherein the comparing comprises one or more of the following comparisons:
a) identifying one or more polymorphism(s) of the detected HPV virus genome and/or genome product and comparing the identified one or more polymorphism(s) to polymorphisms of the reference HPV viral genome, wherein a divergence of 17 or fewer polymorphisms of the detected HPV virus genome and/or genome product as compared to the reference HPV viral genome categorizes the subject as having elevated risk of poor prognosis from de-escalated treatment to the cancer, and wherein a divergence of 18 or more polymorphisms (e.g., non synonymous polymorphisms) to the reference HPV viral genome categorizes the subject as having reduced risk of poor prognosis from de-escalated treatment to the cancer;
b) determining a nearest neighbor distribution of the detected HPV virus genome and/or genome product as compared to the reference HPV viral genome, wherein a nearest neighbor distribution of the sequence information of the detected HPV virus genome and/or genome product less than a predetermined threshold as compared to the reference HPV viral genome categorizes the subject as having elevated risk of poor prognosis from de-escalated treatment to the cancer, and wherein a nearest neighbor distribution equal to or greater than the pre-determined threshold as compared to the reference HPV viral genome categorizes the subject as having reduced risk of poor prognosis from de-escalated treatment to the cancer; and/or
c) calculating a sequence distance of the sequence information of the detected HPV virus genome and/or genome product as compared to the reference HPV viral genome, wherein a sequence distance equal to or less than a pre-determined threshold of the detected HPV virus genome and/or genome product as compared to the reference HPV viral genome categorizes the subject as having elevated risk of poor prognosis from de-escalated treatment to the cancer, and wherein a sequence distance greater than the pre-determined threshold of the detected HPV virus genome and/or genome product as compared to the reference HPV viral genome categorizes the subject as having reduced risk of poor prognosis from de-escalated treatment to the cancer.

9. The method of claim 8, wherein the pre-determined threshold for the nearest neighbor distribution and/or the sequence distance is determined by a phylogenetic analysis of the reference HPV viral genome, wherein the HPV viral genome is a reference library comprising a multitude of reference HPV viral genomes.

10. The method of claim 9, wherein the phylogenetic analysis establishes at least two or more groups, wherein at least one group includes a HPV A1 reference genome, and wherein at least one group is devoid of a HPV A1 reference genome.

11. The method of claim 10, wherein the high risk group is defined as inclusive of any viral genome with a divergence of 17 or fewer polymorphisms as compared to the reference HPV viral genome.

12. The method of claim 10, wherein the high risk group is defined as inclusive of any viral genome with a divergence of 8 or fewer non-synonymous polymorphisms as compared to the reference HPV viral genome.

13. (canceled)

14. The method of claim 8, wherein the one or more polymorphism(s) comprise all polymorphisms, single nucleotide polymorphisms (SNPs), non-synonymous polymorphisms, synonymous polymorphisms, or any combination thereof.

15-16. (canceled)

17. The method of claim 8, wherein identifying one or more polymorphism(s) of the detected HPV virus genome and/or genome product and comparing the identified polymorphisms to polymorphisms of the reference HPV viral genome comprises identifying the presence of one or more E5 I44L, E5 I65V, E2 P219S, L1 T266A, L2 L330F, E6 L90V, E1 S220T, L2 S269P, E2 T310K, E2 I210T, L1 T353P, E2 E232K, E2 A143T, L2 I420T, or E2 N203D polymorphisms or any other polymorphism disclosed herein in the detected HPV virus genome and/or genome product and comparing the identified polymorphisms to the presence of E5 I44L, E5 I65V, E2 P219S, L1 T266A, L2 L330F, E6 L90V, E1 S220T, L2 S269P, E2 T310K, E2 I210T, L1 T353P, E2 E232K, E2 A143T, L2 I420T, or E2 N203D polymorphisms or any other polymorphism disclosed herein in the reference HPV viral genome, wherein a divergence of less than about 8 polymorphisms of the detected HPV virus genome and/or genome product as compared to the reference HPV viral genome categorizes the subject as having elevated risk of poor prognosis from de-escalated treatment to the cancer, and wherein a divergence of greater than about 8 polymorphisms to the reference HPV viral genome categorizes the subject as having reduced risk of poor prognosis from de-escalated treatment to the cancer.

18. The method of claim 6, wherein de-escalated treatment to cancer comprises treating the subject with no and/or a reduced amount of one or more treatments of radiation therapy, chemotherapy, immunotherapy, surgery, and/or intubation of the subject.

19-21. (canceled)

22. The method of claim 6, wherein the subject is receiving and/or has previously received standard treatment for the cancer.

23. The method of claim 6, wherein the subject is not receiving and/or has not previously received standard treatment for the cancer.

24. The method of claim 23, wherein the subject is newly diagnosed has having the cancer.

25. The method of claim 6, wherein the HPV virus genome and/or genome product in the sample comprises a strain of HPV16.

26. The method of claim 6, wherein the reference HPV viral genome comprises a “high risk” HPV viral genome.

27. The method of claim 6, wherein the reference HPV viral genome comprises one or more reference HPV viral genomes.

28. The method of claim 6, wherein the reference HPV viral genome is a reference library comprising the viral genome(s) of HPV strain GenBank® Accession No. NC_001526.4 (HPV16 A1, also numerated as K02718.1), AF536179.1 (HPV16 A2), HQ644236.1 (HPV16 A3), AF534061.1 (HPV16 A4), AF536180.1 (HPV16 B1), KU053915.1 (HPV16 B2), HQ644298.1 (HPV16 B3), KU053914.1 (HPV16 B4), AF472509.1 (HPV16 C1), HQ644244.1 (HPV16 C2), KU053920.1 (HPV16 C3), KU053925.1 (HPV16 C4), HQ644257.1 (HPV16 D1), AY686579.1 (HPV16 D2), AF402678.1 (HPV16 D3), KU053931.1 (HPV16 D4), and/or any combination thereof.

29-31. (canceled)

32. The method of claim 6, wherein the cancer is HPV+ oropharyngeal squamous cell carcinoma (OPSCC).
